(19) United States
(12) Patent Application Publication
(10) Pub. No.: US 2021/0225463 A1
(43) Pub. Date: Jul. 22, 2021

(54) System and Method with Federated Learning Model for Medical Research Applications

(71) Applicant: doc.ai, Inc., Palo Alto, CA (US)

(72) Inventors: James Douglas KNIGHTON, JR., Sunnyvale, CA (US); Philip Joseph DOW, South Lake Tahoe, CA (US); Marina TITOVA, Menlo Park, CA (US); Srivatsa Akshay SHARMA, Palo Alto, CA (US); Walter Adolf DE BROUWER, Los Altos, CA (US); Joel Thomas KAARDAL, San Mateo, CA (US); Gabriel Gabra ZACCAK, Cambridge, MA (US); Sandra Ann R STEYAERT, Izegem (BE)

(73) Assignee: doc.ai, Inc., Palo Alto, CA (US)

(21) Appl. No.: 17/155,078

(22) Filed: Jan. 21, 2021

Related U.S. Application Data
(60) Provisional application No. 62/964,586, filed on Jan. 22, 2020.

(51) Int. Cl. G16H 10/20; G06N 3/08; G16H 40/67; G16H 50/20; G16H 70/60

(52) U.S. Cl. CPC G16H 10/20; G06N 3/08; G16H 70/60; G16H 50/20; G16H 40/67

(57) ABSTRACT

The technology disclosed relates to a system and method of conducting virtual clinical trials. The system comprises a sponsor server configured to specify a target mapping of a clinical trial objective mapper. The target mapping maps participant-specific clinical data to an objective of a virtual clinical trial. The system comprises a plurality of edge devices accessible by respective participants in a plurality of participants. The system comprises a clinical trial conductor server configured to distribute coefficients of the clinical trial objective mapper to respective edge devices to implement distributed training of the clinical trial objective mapper. The clinical trial conductor server is configured to receive participant-specific gradients generated during the distributed training in response to processing participant-specific clinical data. The clinical trial conductor server is configured to aggregate the participant-specific gradients to generate aggregated gradients that cumulatively satisfy the target mapping of the clinical trial objective mapper.

### PRIORITY APPLICATION

This application claims the benefit of U.S. Patent Application No. 62/964,586, entitled “SYSTEM AND METHOD WITH FEDERATED LEARNING MODEL FOR MEDICAL RESEARCH APPLICATIONS,” filed Jan. 22, 2020 (Attorney Docket No. DCAI 1003-1). The provisional application is incorporated by reference for all purposes.

### INCORPORATIONS

The following materials are incorporated by reference as if fully set forth herein:

U.S. Provisional Patent Application No. 62/883,639, titled “FEDERATED CLOUD LEARNING SYSTEM AND METHOD,” filed on Aug. 6, 2019 (Atty. Docket No. DCAI 1014-1);

U.S. Provisional Patent Application No. 62/816,880, titled “SYSTEM AND METHOD WITH FEDERATED LEARNING MODEL FOR MEDICAL RESEARCH APPLICATIONS,” filed on Mar. 11, 2019 (Atty. Docket No. DCAI 1008-1);

U.S. Provisional Patent Application No. 62/481,691, titled “A METHOD OF BODY MASS INDEX PREDICTION BASED ON SELFIE IMAGES,” filed on Apr. 5, 2017 (Atty. Docket No. DCAI 1006-1);

U.S. Provisional Patent Application No. 62/671,823, titled “SYSTEM AND METHOD FOR MEDICAL INFORMATION EXCHANGE ENABLED BY CRYPTO ASSET,” filed on May 15, 2018;

Chinese Patent Application No. 201910235758.60, titled “Joint learning model systems and methods, apparatus, and computer-readable storage media,” filed on Mar. 27, 2019;

Japanese Patent Application No. 2019-097904, titled “SYSTEM HAVING COMBINED LEARNING MODEL FOR MEDICAL RESEARCH APPLICATIONS, AND METHOD,” filed on May 24, 2019;

U.S. Nonprovisional patent application Ser. No. 15/946,629, titled “IMAGE-BASED SYSTEM AND METHOD FOR PREDICTING PHYSIOLOGICAL PARAMETERS,” filed on Apr. 5, 2018 (Atty. Docket No. DCAI 1006-2);

U.S. Nonprovisional patent application Ser. No. 16/167,338, titled “SYSTEM AND METHOD FOR DISTRIBUTED RETRIEVAL OF PROFILE DATA AND RULE-BASED DISTRIBUTION ON A NETWORK TO MODELING NODES,” filed on Oct. 22, 2018; and

U.S. Nonprovisional Patent Application No. 63/064,624, titled “SYSTEMS AND METHODS FOR VIRTUAL CLINICAL TRIALS,” filed on Aug. 12, 2020 (Atty. Docket No. DCAI 1013-1).

### FIELD OF THE TECHNOLOGY DISCLOSED

The disclosed system and method are in the field of machine learning. To be more specific, in the field of federated machine learning utilizing computation capability of edge devices and a federated learning (“FL”) aggregator, which is typically cloud-based, relative to the edge devices. In this context, edge devices typically are mobile devices, but also can include nodes that aggregate data from multiple users.

### BACKGROUND

The subject matter discussed in this section should not be assumed to be prior art merely as a result of its mention in this section. Similarly, a problem mentioned in this section or associated with the subject matter provided as background should not be assumed to have been previously recognized in the prior art. The subject matter in this section merely represents different approaches, which in and of themselves can also correspond to implementations of the claimed technology.

Traditional software (1.0) uses declarative inputs and follows deterministic trees of logic, but machine learning (2.0) deals with noisy inputs and uses probabilities. Since the beginning of epistemology, there have been two theories, top-down (Plato theory) and bottom-up (Aristotle theory). Top-down deep learning starts from a theory, not from the data. Bayesian logic combines generative models and probability theory to calculate just how likely it is that the particular answer is true given the data. Bottom-up deep learning starts from the data, not the theory. It consists of labeling large amounts of data (both “right” and “wrong” data) to determine association and build a foundation for pattern recognition. It can even learn unsupervised, detecting patterns in data with no labels at all and identify clusters (factor analysis).

The year of 2013 to 2016, the era of the renewed interest in machine learning technology, was followed by the era of deep learning technology, spanning 2016 to the priority filing of this disclosure in 2019. 2019 leads us to the next deep dive of intelligent and/or neuromorphic computing, the federated learning technology.

With machine learning, humans enter input examples and desired output, sometimes called ground truth, and a system learns. Thereafter, output comes from a trained classifier or network. The classifier or network does not have to be programmed directly, but the semantics by which it is generated are programmed. This way, humans train a classifier or network to encode complex behavior with parameters that can be thought of as rules of low complexity. Although the algorithm does not need to be programmed, these neuron networks still need to be trained by humans. They need the input data to be presented in a structured way. Hence, there is a lot of human-aided labor involved in collecting, cleaning, and labeling data. Human talent also is applied to evaluating a model and steering its training in the right direction.

Deep learning applies multi-layered networks to data. While training can be automated, there remains the problem of assembling training data in the right formats and sending data to a central node of computation with sufficient storage and compute power. In many fields, sending personally identifiable, private data to any central authority causes worries about data privacy, including data security, data ownership, privacy protection and proper authorization and use of data. Clinical trials are an important part of health care research that involve participants who provide data or feedback to determine efficacy of a new treatment protocol or a new drug, etc. Clinical trials are expensive to conduct as participants need to go to a clinic or a pharmacy to participate in the study. Maintaining privacy of participants' clinical data is important for success of clinical trials.

An opportunity arises to apply federated learning to conduct clinical trials and train high performance machine learning models by utilizing different data sources without breaking the privacy regulations and laws.

### BRIEF DESCRIPTION OF THE DRAWINGS

The patent or application file contains at least one drawing executed in color. Copies of this patent or patent application publication with color drawing(s) will be provided by the Office upon request and payment of the necessary fee. The color drawings also may be available in PAIR via the Supplemental Content tab.

FIG. 1 is a high-level architecture of a system that can be used to conduct clinical trials.

FIG. 2 is a flow chart illustrating an example core template of machine learning workflow, consistent with embodiments of the present disclosure.

FIG. 3 is a diagram illustrating an example federated learning model with multiple edge devices and a clinical trial conductor server.

FIG. 4A is a diagram illustrating an example use case of a federated learner system, comprising one-to-many tensors for distributed clinical trials.

FIG. 4B is a diagram illustrating an example use case of a federated learner system, comprising federated learners (or Fleas) at edge devices for distributed clinical trials.

FIG. 5 is a diagram illustrating an example clinical trial conductor server or Federated Learning (FL) aggregator.

FIG. 6 is a diagram illustrating an example use case of tensor globalization of a federated learner system.

FIG. 7A and FIG. 7B are diagrams illustrating an example use case of a federated learner system in a linear training trial and in an adaptive and continuously learning distributed trial, comprising federated learners and FL aggregator for application of data trial.

FIG. 8 is a diagram illustrating an example use case of a federated learner system, comprising simulated control arms for trials.

FIG. 9 is a diagram illustrating centralized data collection and training, leading to deployment to edge devices.

FIG. 10 is a diagram illustrating edge device update training followed by centralized aggregation of the updated models.

FIG. 11 is a diagram illustrating more detail of data at edge devices during update training.

FIG. 12 is an example of conducting a clinical trial for allergy risk prediction using the federated learning system.

FIG. 13 is an example convolutional neural network (CNN).

FIG. 14 is a block diagram illustrating training of the convolutional neural network of FIG. 13.

FIG. 15 is a simplified block diagram of a computer system that can be used to implement the technology disclosed.

The patent or application file contains at least one drawing executed in color. Copies of this patent or patent application publication with color drawing(s) will be provided by the Office upon request and payment of the necessary fee. The color drawings also may be available in PAIR via the Supplemental Content tab.

### DETAILED DESCRIPTION

The following discussion is presented to enable any person skilled in the art to make and use the technology disclosed, and is provided in the context of a particular application and its requirements. Various modifications to the disclosed implementations will be readily apparent to those skilled in the art, and the general principles defined herein may be applied to other implementations and applications without departing from the spirit and scope of the technology disclosed. Thus, the technology disclosed is not intended to be limited to the implementations shown, but is to be accorded the widest scope consistent with the principles and features disclosed herein.

### INTRODUCTION

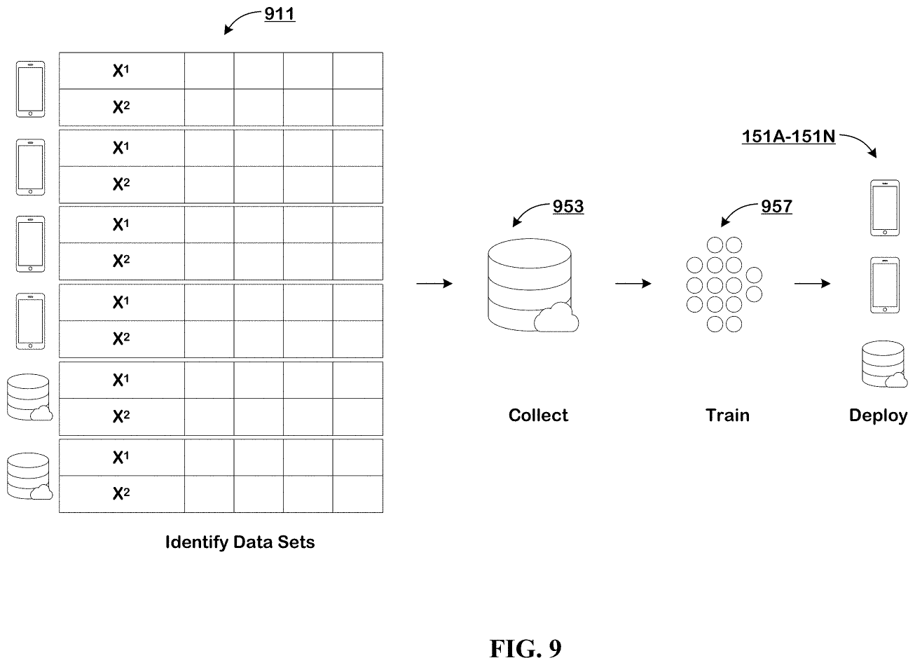

Traditionally, to take advantage of a dataset using machine learning, all the data for training had to be gathered to one place. A typical machine learning workflow is illustrated by FIG. 9. Having identified a problem space and a learning task, one finds a large body of data 911, 953 to train a model at a central repository, in a centralized manner (957). After being satisfied with the model, one deploys it to edge devices 151A-151N or to a cloud-based compute resource for prediction. Typical model training involves centrally collecting the data and centrally training the model even when it is deployed in a distributed manner. This involves bringing the data 911 to a central repository 953 to gain control over how it's used in training 957. However, as more of the world becomes digitized, this can fail to scale with the vast ecosystem of potential data sources that could augment machine learning (ML) models in ways limited only to the imagination. To solve this, we resort to federated learning (“FL”).

Federated Learning

Federated learning (FL) approach aggregates model weights across multiple devices without such devices explicitly sharing their data. In this disclosure we use horizontal federated learning which assumes a shared feature space, with independently distributed samples stored on each device. FL is a set of techniques to perform machine learning on distributed data—data which may lie in highly different engineering, economic, and legal (e.g., privacy) landscapes. In the literature, it is mostly conceived as making use of entire samples found across a sea of devices (i.e., horizontally federated learning), that never leave their home device. The ML paradigm remains otherwise the same.

End users use application programs on their respective devices (also referred to as edge devices) by which end users collect data, train, compute, and evaluate data stored in devices these application programs run on. No data leaves devices where it is stored and computed. Devices later federate data globally by sending “derived insights,” technically a bunch of tensors, to a computing cloud where all these derived insights are averaged. Devices then receive from the computing cloud an updated matrix which can improve local prediction of these devices. The improved local prediction again improves derived insights as updates. With federated learning, a device on the edge can send potentially de-identified updates to a model instead of having to accept the burden of sending over the entirety of its raw data in order for the model to be updated. As a result, federated learning greatly reduces privacy concerns since the data never leaves these devices, just an encrypted, perturbed gradient of data leave. Federated learning further greatly reduces ownership concerns as end users are enabled to opt in or out to share updates created in devices. Federated learning further greatly reduces security concern, because there is no single point of failure overall to the whole system, and hackers cannot hack millions of phones one by one. We use federated learning to conduct clinical trials or studies as described in the following sections.

Virtual Clinical Trials

Clinical research is medical research involving people. There are two types of clinical research, observational studies and clinical trials. Observational studies observe people in normal settings. Researchers gather information, group volunteers according to broad characteristics, and compare changes over time. These studies may help identify new possibilities for clinical trials. Clinical trials are research studies performed in people that are aimed at evaluating a medical, surgical, or behavioral intervention. They are the primary way that researchers find out if a new treatment, like a new drug or diet or medical device (for example, a pacemaker or a face mask for sleep apnea) is safe and effective in people. Several parties are involved in clinical trials such as a principal investigator (PI) and her team who sponsor a clinical trial or study, participants, a pharmacy or a distributor or a contract research organization (CRO) that can distribute drugs or medical devices to participants on behalf of the PI and her team.

Clinical trials are expensive to conduct and require considerable planning and effort. The participants may be divided into two or more groups. The distribution of participants in groups maybe random. One or more groups may be given the experimental drugs or medical devices that are being investigated. This group of participants is referred to as an intervention group or a real group. A group of participants can be provided a fake treatment which is also referred to as a placebo. A placebo is defined as any therapy used for its nonspecific, psychological, or psychophysiological effect and is without specific activity of the condition being treated (Shapiro & Morrison, 1978, “The Placebo Effect in Psychotherapy and Behavior Change”). As participants do not like to visit hospitals or clinics to avoid getting infected by viruses such as COVID-19, the researchers are motivated to conduct clinical trials in a virtual or siteless environment.

Virtual or siteless clinical trials can be conducted without any physical interaction between the parties. In addition, the virtual clinical trials are convenient for participants and researchers to setup and conduct the trial. Participants can easily enroll in a clinical trial using a software application. However, to conduct the clinical trial at various levels of privacy (such as a single blinded, double blinded, etc.), it is required that the entire setup and data collection steps of the trial be conducted in a blinded manner. That is the sponsor may not know the assignment of participants to clinical trial groups and/or the details of the data collected from each participant. The intermediary such as a contract research organization may handle the collection of data from the participants and provide analytics, aggregated data, or a trained prediction model to sponsor.

In the present disclosure, system and method for federated learning applied to conduct virtual clinical trials is presented. We apply federated learning to conduct a virtual or siteless clinical trial. In this application of federated learning, a sponsor server represents the principal investigator and her team. The sponsor server can be coupled to a communication network. The sponsor server is configured to specify a target mapping or target configuration of a clinical trial objective mapper. The target mapping is a prediction task for the clinical trial objective mapper (or machine learning model such as a neural network). The target mapping maps participant-specific clinical data to an objective of a virtual clinical trial. The participant-specific clinical trial data is the input to the clinical trial objective mapper and is entered by the respective participants on their edge devices. Examples of objective of a clinical trial can include predicting a disease, predicting symptom of disease, or predicting efficacy of a treatment, etc.

A clinical trial conductor server can represent the CRO when conducting the virtual clinical trials. The clinical trial conductor server can be interposed between the edge devices of participants of the clinical trial and the sponsor server. The clinical trial conductor server can distribute coefficients of the clinical trial objective mapper to respective edge devices in the plurality of edge devices to implement the distributed training of the clinical trial objective mapper. The clinical trial objective mapper can receive participant-specific gradients from the respective edge devices. The participant-specific gradients are generated during distributed training of the clinical trial objective mapper in response to processing participant-specific clinical data through the coefficients of the clinical trial objective mapper at the respective edge devices. The clinical trial conductor server can aggregate the participant-specific gradients to generate aggregated gradients that cumulatively satisfy the target mapping of the clinical trial objective mapper. We present a summary of the invention followed by details of the environment to conduct virtual clinical trials using federated learning.

Fundamentally, a simple algorithm is based when a model of a process to arrive at increasingly complex skills in machines and the inner workings are called. First, a cost function, e.g., how well the network solves the problem, which you want to minimize, is defined. Secondly, the network is run once and see how it does at that cost function. Thirdly, the values of the connections are adjusted, and the network is run again. Fourthly, the difference between these two results is the direction or slope in which the network moved between the two trials. This process is called a gradient. Fifthly, if the slope is downhill the connections are changed in downhill direction, and if the slope is uphill, the connections are changed in the opposite direction. Steps three to five are repeated until there is no improvement in any direction. It means the system with gradient descent is optimized and has arrived at the limits of it, what is call the local minimum. To get out of the local minimum, the values are randomly changed using stochastic gradient dissent. Eventually after many times mining the computational universe for the right gradients, a deeper or even global minimum value is arrived.

Generally provided is a system for federated learning utilizing computation capability of edge devices and cloud. The system comprises multiple edge devices of end users, one or more federated learner update repository, and one or more cloud. Each edge device comprises a federated learner model, configured to send tensors to federated learner update repository. Cloud comprises a federated learner model, configured to send tensors to federated learner update repository. Federated learner update repository comprises a back-end configuration, configured to send model updates to edge devices and cloud.

Generally provided is a method of federated learning utilizing computation capability of edge devices and cloud. The method comprises sending out tensors by multiple edge devices and/or clouds with federated learning models, receiving tensors by a federated learning update repository from the multiple edge devices with federated learning models, sending out model updates from the federated learning update repository, and receiving model updates by multiple edge devices and/or clouds with federated learning models.

Generally provided is a federated learning system comprising multiple federated learners or Fleas, whereas each federated learner is configured to be an end user side library, built for an edge device environment. Such federated learner update model calculations from data collected in the edge device, model post-processing and, model sharing with a central federated learner update repository, download of updated models, and model evaluation with sharing of evaluation metrics similar to that of model updates.

Generally provided is a federated learner update repository (or an FL aggregator, federated learning repository, some other central authority or compute resource) comprising a federated learning back-end configuration responsible to collect model updates and evaluations sent from Flea end users which requires high availability, organize models that can be updated from end user side updates along with the operations required to perform these updates, admit or reject proposed updates from each end user based on criteria and metadata sent by end user, aggregate admissible end user updates into a single update to each model, redistribute updated models to the end user side if with availability, and report aggregations of model evaluations based on similar admissibility criteria as those used in updates.

This summary is provided to efficiently present the general concept of the invention and should not be interpreted as limiting the scope of the claims.

We now present an overview of the system to conduct virtual clinical trials using federated learning technology.

Environment

Many alternative embodiments of the present aspects may be appropriate and are contemplated, including as described in these detailed embodiments, though also including alternatives that may not be expressly shown or described herein but as obvious variants or obviously contemplated according to one of ordinary skill based on reviewing the totality of this disclosure in combination with other available information. For example, it is contemplated that features shown and described with respect to one or more embodiments may also be included in combination with another embodiment even though not expressly shown and described in that specific combination.

For purpose of efficiency, reference numbers may be repeated between figures where they are intended to represent similar features between otherwise varied embodiments, though those features may also incorporate certain differences between embodiments if and to the extent specified as such or otherwise apparent to one of ordinary skill, such as differences clearly shown between them in the respective figures.

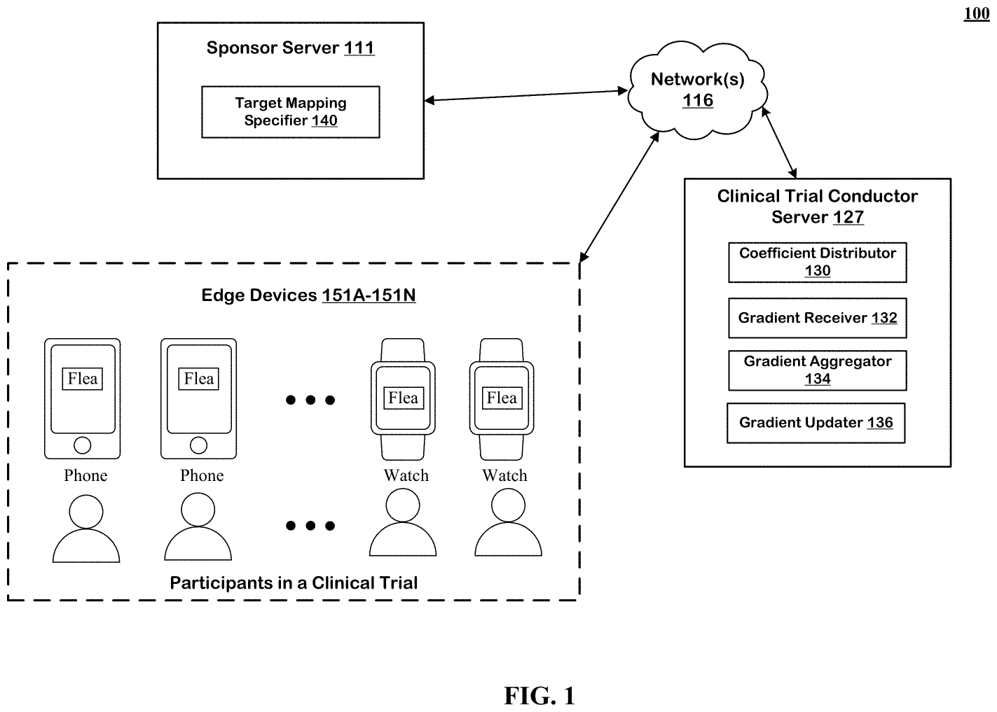

We describe a system for conducting virtual clinical trials with. The system is described with reference to FIG. 1 showing an architectural level schematic of a system 100 in accordance with an implementation. Because FIG. 1 is an architectural diagram, certain details are intentionally omitted to improve the clarity of the description. The discussion of FIG. 1 is organized as follows. First, the elements of the figure are described, followed by their interconnection. Then, the use of the elements in the system is described in greater detail.

FIG. 1 includes the system 100. This paragraph names labeled parts of system 100. The figure includes a sponsor server 111, a clinical trial conductor server 127, edge devices 151A-151N for respective participants.

A network(s) 116 couples the sponsor server 111, the clinical trial conductor server 127, and edge devices 151A-151N. The edge devices 151A-151N of respective participants can have an application (or app) that can be used to communicate to the clinical trial conductor server 127. The app can collect clinical trial data and store it locally on the edge devices of respective participants.

The sponsor server 111 can include a target mapping specifier 140 that can include logic to specify a target mapping of the clinical trial objective mapper. The target mapping maps participant-specific clinical data to an objective of a virtual clinical trial. The clinical trial conductor server 127 can include a coefficient distributor 130, a gradient receiver 132, a gradient aggregator 134, and a gradient updater 136. The coefficient distributor 130 includes logic to distribute coefficients of the clinical trial objective mapper to respective edge devices of participants to implement distributed training of the clinical trial objective mapper. The gradient receiver 132 includes logic to receive participant-specific gradients from the respective edge devices. The participant-specific gradients are generated during the distributed training in response to processing participant-specific clinical data through the coefficients of the clinical trial objective mapper at the respective edge devices. The gradient aggregator 134 includes logic to aggregate the participant-specific gradients to generate aggregated gradients that cumulatively satisfy the target mapping of the clinical trial objective mapper. The gradient updater 136 includes logic to apply aggregated gradients to coefficients of the clinical trial objective mapper to generate updated coefficients of the clinical trial objective mapper. The updated clinical trial objective mapper can be distributed to the edge devices of the respective participants to improve the performance of the machine learning model.

The system can also include one or more databases. For example, the system can include a database to store participant-specific gradients, or aggregated gradients, etc. The system can also include a clinical trial setup database that can contain setup data or configurations for the clinical trials. The setup data can specify values of various parameters for the clinical trial, such as participant selection criteria, assignment ratio of participants to different treatment groups, etc. The principal investigator can set values for different parameters of clinical trial. For example, the ratio of participants to be assigned to each group in the clinical trial. The setup data can also indicate the level of data privacy (e.g., single blinded, double blinded, triple blinded, etc.) for the clinical trial.

Completing the description of FIG. 1, the components of the system 100, described above, are all coupled in communication with the network(s) 116. The actual communication path can be point-to-point over public and/or private networks. The communications can occur over a variety of networks, e.g., private networks, VPN, MPLS circuit, or Internet, and can use appropriate application programming interfaces (APIs) and data interchange formats, e.g., Representational State Transfer (REST), JavaScript Object Notation (JSON), Extensible Markup Language (XML), Simple Object Access Protocol (SOAP), Java Message Service (JMS), and/or Java Platform Module System. All of the communications can be encrypted. The communication is generally over a network such as the LAN (local area network), WAN (wide area network), telephone network (Public Switched Telephone Network (PSTN), Session Initiation Protocol (SIP), wireless network, point-to-point network, star network, token ring network, hub network, Internet, inclusive of the mobile Internet, via protocols such as EDGE, 3G, 4G LTE, Wi-Fi and WiMAX. The engines or system components of FIG. 1 are implemented by software running on varying types of computing devices. Example devices are a workstation, a server, a computing cluster, a blade server, and a server farm. Additionally, a variety of authorization and authentication techniques, such as username/password, Open Authorization (OAuth), Kerberos, Secured, digital certificates and more, can be used to secure the communications.

### Federated Learning Process Flow

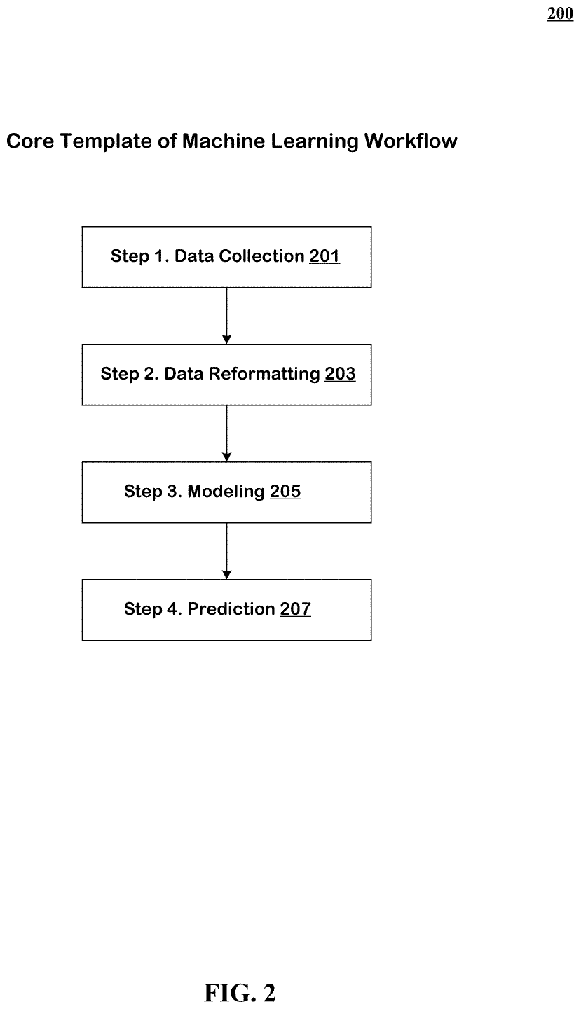

FIG. 2 is a high-level flow chart 200 of machine learning workflow. In some embodiments, a core template of machine learning workflow comprises four steps. Step 1 is data collection 201, to procure raw data. Step 2 is data re-formatting 203, to prepare the data in the right format. Step 3 is modeling 205, to choose and apply a learning algorithm. Step 4 is predictive analytics 207, to make a prediction. Variables that are likely to influence future events are predicted. Parameters used to make the prediction are represented in multi-dimensional matrix, called tensors.

A multi-dimensional matrix, or tensor, has certain features commend this data representation to machine learning. Linear algebra operations are efficiently applied by GPUs and other parallel processors on computers. Linearization or differentiation make it feasible to frame optimization problems as linear algebra problems. Big data is difficult to process at scale without tensors, so many software tools have come onto market that simplify tensor computing, e.g., TensorLab, Matlab package, Google TensorFlow, etc. Hardware is following software. Groups of engineers are working on tensor processing accelerator chips, e.g., NVDIA GPUs, Google TPUs (Tensor Processing Units), Apple A11, Amazon Inferentia, Graviton and Echo-chip, Facebook Glow, and a whole range of technology companies that make Application-Specific Integrated Circuits (ASIC), field programmable gate arrays (FPGAs) and coarse-grained reconfigurable arrays (CGRAs) adapted to calculate tensors with tensor calculation software.

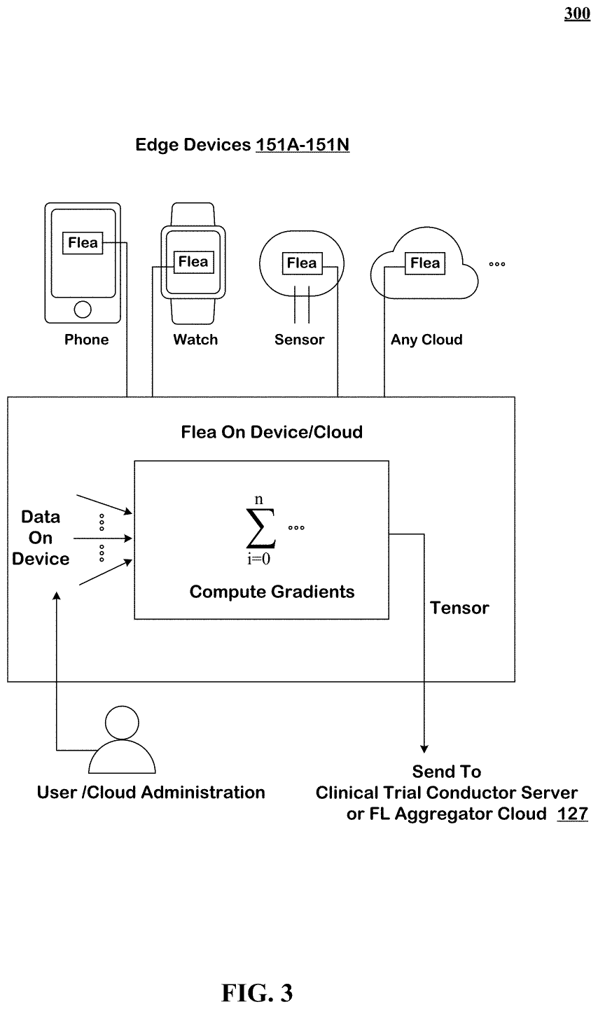

FIG. 3 is a diagram 300 illustrating an example federated learning model with multiple edge devices and a central FL aggregator.

In some embodiments, the system comprises multiple edge devices 151A-151N of clinical trial participants (end users), a cloud, and the clinical trial conductor server 127 (or FL aggregator). The multiple edge devices can be coupled to the communication network. Each edge device can comprise a memory, configured to store end user data and a federated learner, configured to send gradients (or tensors), wherein such gradients (or tensors) are calculated based on the end user data stored in the memory. The cloud is electrically coupled to the communication network, comprising a federated learner, configured to send gradients (or tensors), wherein such gradients (or tensors) are calculated based on data available in public domain. The clinical trial conductor server is configured to receive gradients (or tensors) from edge devices and cloud and send out updated gradients (or updated models) to edge devices and cloud.

A federated learner (Flea) can be implemented as an end user side library, built for an edge device environment, to perform local model update calculations using data collected in the edge device environment. The Flea can perform post-processing after model updating, including applying perturbations (e.g., encryption and introduction of noise for privacy purposes), sharing the model update with a central update repository (i.e., an FL aggregator), optionally downloading updated models, evaluating updated models, and sharing evaluation metrics across platforms, e.g., Flea-iOS (for iPhones), Flea-Android (for Android phones), Flea-kubernetes (for node clients), etc.

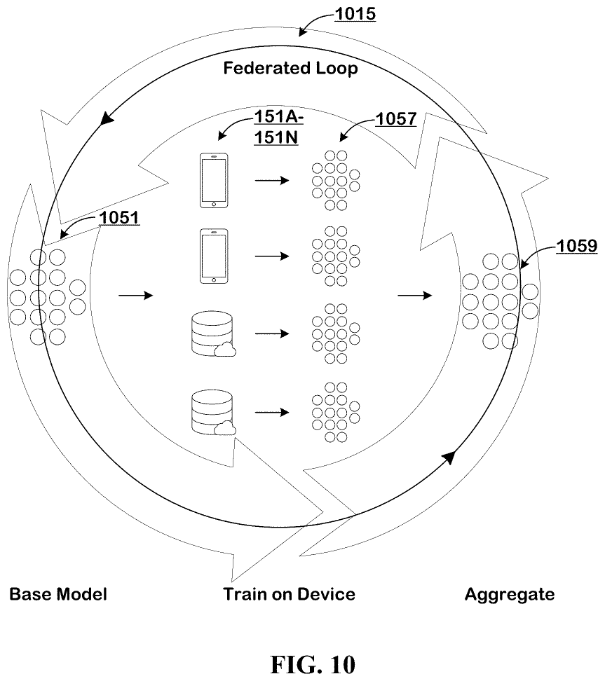

We refer to FIG. 10, presenting a federated loop 1015 to explain the federated learning workflow. In a federated workflow, we start with a base model 1051 that may have been trained in this conventional manner. Once this base model 1051 is trained, refinement can proceed without centrally collecting any further data. Instead, the base model 1051 is distributed to individual edge devices 151A-151N. These edge devices perform local training to generate local model updates 1057, using data (not shown) that is on those devices. The federated workflow aggregates the local updates into a new global model 1059 which will become our next base model 1051 that will be used for inference and additional rounds of training a federated loop 1015. Again, updating via the federated loop 1015 does not require centrally collecting data. Instead, we're sending the model to the data for training, not bringing data to the model for training. This is a decentralized workflow instead of a centralized workflow.

Flea end users (or participants) can communicate and collaborate with one another (potentially in tandem with one or more FL aggregator backends) to build and update models of computation in multiple ways. These configurations are described in the context of medical research use cases.

In some embodiments, Flea end users (or participants), via respective edge devices, can communicate and collaborate with one another to build and update models of computation in lateral tensor ensembles in a one-to-one manner. The end users can also laterally organize their own trials and choose a central cloud directory to send the gradients and get the averaged or aggregated gradients back in a distributed fashion.

In yet some other embodiments of the disclosure, tensors (or gradients) can be configured to function tensorial handshakes, with one-to-one tensors for distributed clinical trials. Participants can also laterally organize their own trials and choose a central cloud directory to send the gradients and get the averaged or aggregated gradients back in a distributed fashion.

In some embodiments, Flea end users or participants, via respective edge devices, can communicate and collaborate with one another to build and update models of computation in tensor economy in a many-to-one manner. When conducting virtual clinical trials, each end user or participant can be called upon by several sponsors to conduct several trials at the same time and can use the same underlying data to create new tensors.

In another embodiment, there can be many-to-one tensors for distributed clinical trials. Each participant can be called upon by several sponsors to conduct several data trials at the same period of time.

In some embodiments, Flea end users or participants, via respective edge devices, can communicate and collaborate with one another to build and update models of computation in autonomous tensor ensembles in a many-to-many manner. Just as algorithms start to write themselves, devices without human intervention will start to collect information between each other. These will just behave like many insect species, including ants and bees, who work together in colonies, and their cooperative behavior determines the survival of the entire group. The group operates like a single organism, with each individual in a colony acting like a cell in the body and becomes a “superorganism.” Federated Deep learning only needs these small players like insects, ants, critters, and bees to create big and smart things with immense, complex, and adaptive social power and ambitious missions.

In some embodiments, Flea end users or participants, via respective edge devices, can communicate and collaborate with one another to build and update models of computation in vertical tensor ensembles in a one-to-many manner. With federated learning, a global protocol is sent from one central authority to many participants who collect information on their edge device, label the information and compute it locally, after which they sent the tensors to the central cloud of the sponsor. They aggregate all the tensors (or gradients) and then report the updated and averaged tensors (gradients) back to each of the participants.

We now present details of using federated learning in health care space. This is followed by discussion regarding application of federated learning to conduct virtual clinical trials.

### Health Care Space

Federated Learning be particularly helpful when dealing with sensitive data, such as medical information in the health care space. In this space, there are a number of issues around data sensitivity. It is crucial to address privacy, both to attract participation of individuals who are reluctant to share sensitive medical information and to comply with regulations.

In some circumstances, an individual may understand the research value of sharing information but doesn't trust the organization that they're being asked to share with. The individual may wonder what third parties that could gain access to their data. On the B2B side, there are intellectual property issues that thwart companies that want to collaborate but are unable to share their raw data for IP reasons. The technology disclosed can enable collaboration without necessarily sharing data. Also, on the B2B side, some companies have internal data policies that prevent even intra-company, cross-division sharing of data. These companies would benefit from collaboration without data sharing.

In the health care space, regulatory concerns can be paramount. The United States has the federal Health Insurance Portability and Accountability Act, HIPAA. The Eurozone has General Data Protection Regulation or GDRP. Both impose strict rules around how medical data is handled and shared.

The technology disclosed applies federated learning to an environment where it is difficult to share underlying data due to data sensitivity concerns. The technology disclosed focuses on so-called horizontal federated learning, in which devices have a different sample space for the same feature space, as opposed to vertical learning, which can be applied to the same sample space with different feature spaces. Horizontal learning applies well to a mobile environment, where a model can be completely shared.

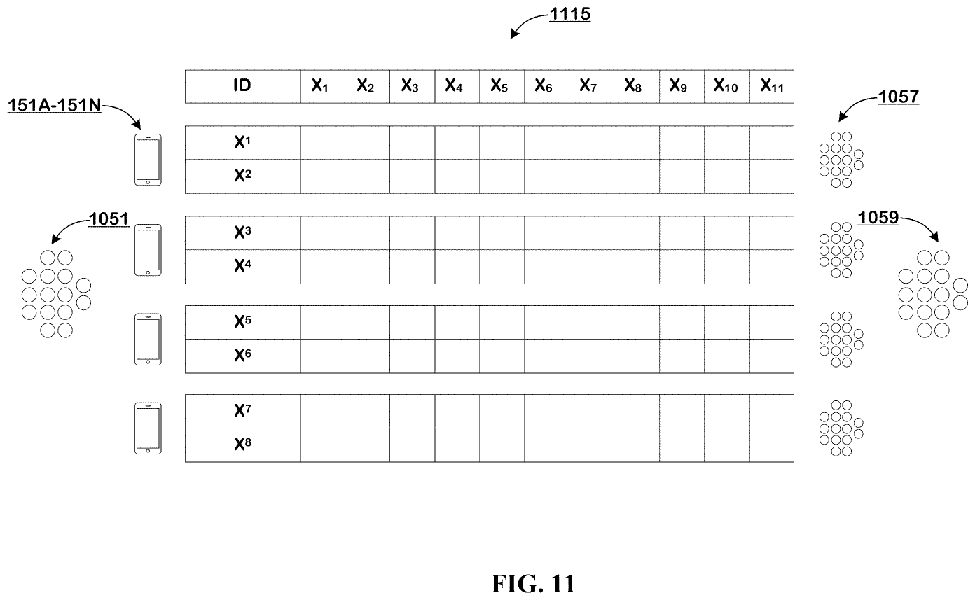

Consider, with reference to FIG. 11, a data set in the form of a table 1115. This data can be visualized as is a matrix with samples across rows and features down columns. The rows of data may correspond to samples used with a neural network for training. They also may correspond to a SQL-returned table and may have a unique identifier, IDs, across rows and again have columns of features. In FIG. 11, the dataset 1115 is divided horizontally among edge devices 151A-151N. In this horizontally partitioned dataset, each subset of the data has access to the same feature space, but has its own sample space, as one can imagine of data trained or collection on mobile phones.

Consider an image processing application and a tensor applied to images that are, for example, 224×224 pixels, prior to being sent to a neural network for inference and training by backward propagation. Images on different devices have the same feature space, but they're different images, belonging to different sample spaces. Each edge device can start with the same base model. An FL aggregator or federated learning repository or some other central authority or compute resource sends the base model to the edge device for update training, to produce updated models 1057. The edge devices 151A-151N train using respective partitions of the data 1115, producing the updated models 1057, which are aggregated 1059 into an updated model which can be distributed as a new base model 1051. In this process, the base model resides locally on each device. Each device trains locally on data that is available on device. The federated loop aggregates the local updates to produce a new global model. We now present application of federated learning to conduct virtual clinical trials.

Clinical Trials

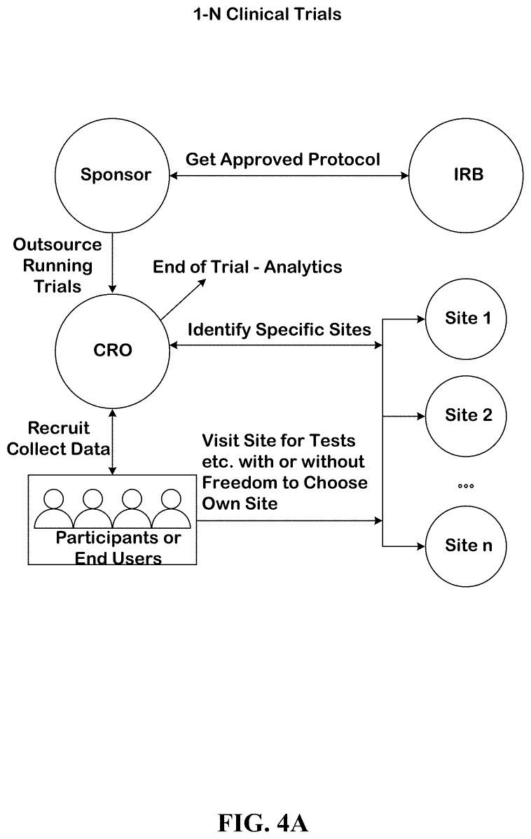

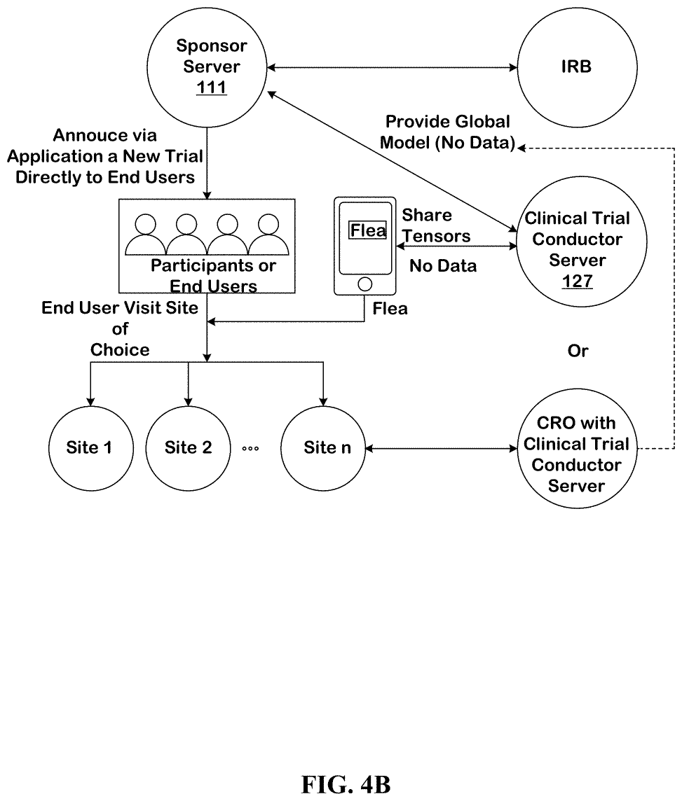

FIG. 4A is a diagram illustrating an example use case of a traditional clinical trial where the one-to-many tensors for distributed clinical trials could be applied.

In some embodiments, tensor ensembles are vertical in a one-to-many structure, called Vertical Tensor Ensembles. Most clinical trials are centralized which consist of one sponsor who centrally produces the protocol and uses several sites where many end users can go for physical exams and laboratory tests. This procedure is time consuming and costly and mostly outsourced to Contract Research Organizations (CROs). With Federated Learning a global protocol is sent from one central authority (such as sponsor server 111) to many end users who collect information on their edge devices, e.g., smartphones, label the information and compute it locally, after which the outcome tensors are sent to the central FL aggregator (or the clinical trial conductor server 127) of the sponsor. The clinical trial conductor server aggregates all the tensors and then reports the updated and averaged tensors back to each of the end users. This one-to-many tensors are configured to conduct distributed clinical trials.

FIG. 4B is a diagram illustrating an example of using a federated learner system to conduct one-to-many tensor exchanges for distributed clinical trials, using so-called Fleas.

In some embodiments, sponsor of a digital clinical trial, typically a data trial, announces the data trial directly to participants or end users via application program installed on end users' devices. Each end user device includes a federated learner. The federated learners are configured to share tensors with a centralized FL aggregator or the clinical trial conductor server. The or the clinical trial conductor server is configured to share with the sponsor only a global model, not data or model updates from individual participant.

In some embodiments, sponsor of a data trial announces the trial directly to participants or end users. Participants are free to choose from many specific sites to participate the data trial. Each of these specific sites are configured to be connected with a CRO which holds or has access to the clinical trial conductor server. Similarly, federated learners of devices are configured to share tensors on data with the CRO FL aggregator. The CRO centralized FL aggregator is configured to share with the sponsor only a global model, not data or model updates from individual participants.

Both of these embodiments, comparing to traditional clinical trial procedure involving Institutional Review Board (IRB), improve the efficiency of clinical trials drastically. End users enjoy far better flexibility of participating clinical trials. The one-to-many trials reduce the need for a CRO from a data management perspective for Pharmaceutical company. End users are not sharing data, just trained models' weights. End users have the option to go to preferred site of choice, instead of being limited to a chosen and assigned site to them. This also means more virtual trials are possible without introducing data quality issues. The clinical trial conductor server intermediary, either a centralized server or a CRO having licensed the clinical trial conductor server, can do the global averaging of the weights. A sponsor, such as a pharmaceutical company or the PI, does not do the global averaging of the weights, thus removing doubts of any bias by the sponsor. The audits are on the weights and algorithms, thus removing most human bias in checking data quality.

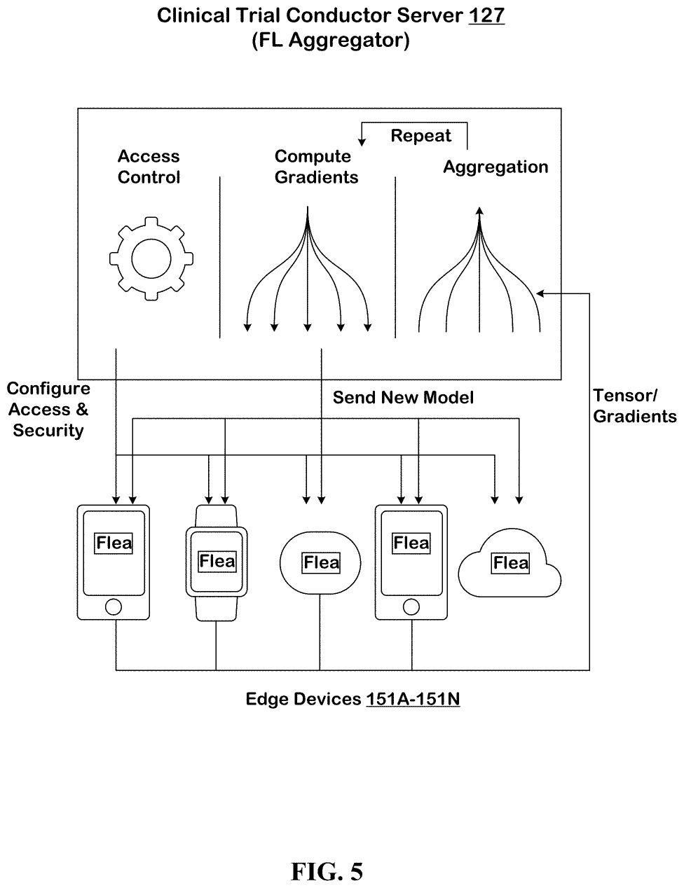

FIG. 5 is a diagram illustrating an example or the clinical trial conductor server. In this example, Flea is configured to be embedded in various edge devices belonging to end users. Such edge devices can be but not limited to any electronic device which is capable of connecting to internet or similar web. For example, mobile phones, smart watches, sensor modules in car or home, or a cloud server, etc.

An FL aggregator or the clinical trial conductor server is designed as a federated learning back-end responsible to collect model updates and evaluations sent from Flea end users (or participants) which requires high availability. The clinical trial conductor server can organize models that can be updated from end user side updates along with the operations required to perform these updates, admit, or reject proposed updates from each end user based on criteria such as history of end user's submissions (e.g., an end user's credibility score) as well as end user sent metadata. The clinical trial conductor server aggregates admissible end user updates into a single update to each model and redistributes updated models to the end user side. The clinical trial conductor server reports aggregations of model evaluations based on similar admissibility criteria as those used in updates. It conducts tensorial handshakes, which are protocols that govern the exchange of information between federated learners running on end user (or participants') devices and the FL aggregator, or amongst collectives of federated learners, on the initiative of end users themselves.

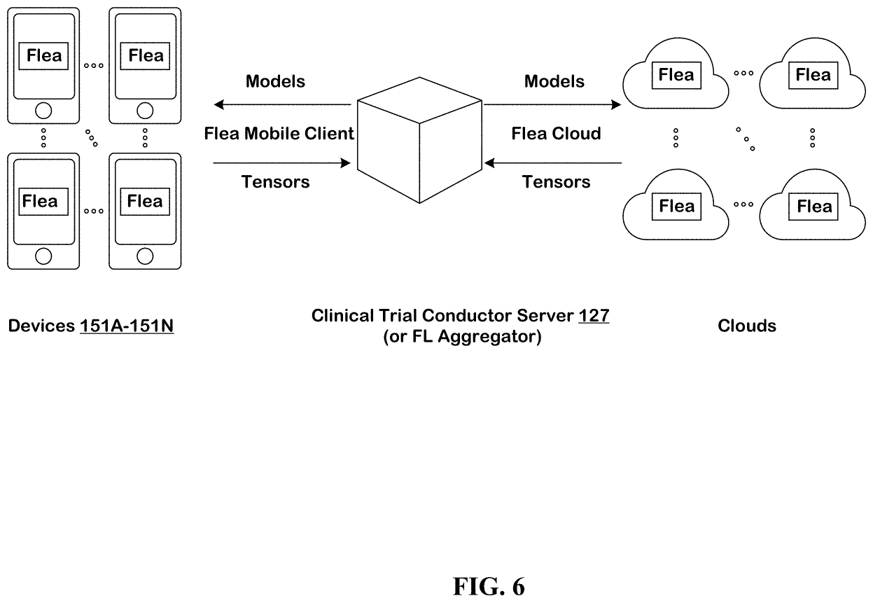

FIG. 6 is a diagram illustrating an example use case of tensor globalization of a federated learner system. Consider the example of a biotech company that has a federated learner model trained for Parkinson's disease. Traditionally, most clinical trials are centralized. They consist of one sponsor who centrally produces the protocol and uses several sites where the many participants can go for exams and tests. This procedure is time consuming and costly and mostly outsourced to Clinical or Contract Research Organizations (CROs).

New alternatives that are now becoming available as the technology disclosed, which manipulate tensors as proxies for data, evolve. The distributed structure of a clinical trial, instead of flat, can be curved into an n-dimensional manifold or surface. This also changes the nature of models. Models themselves are simply tensor ensembles. As edge computational units become more powerful, each computational unit on the edge can house its own model. Both data-derived tensors and model ensembles can be freely exchanged between edge devices or units as shown by broken line connections between devices 151A-151N.

The clinical trial conductor server is configured to be provided at least a federated learner model and a multi-dimensional matrix. The tensors coming out of that model can be averaged with the tensors of biotech model. The biotech company gets the global model back. The trained model can then be applied to data collected from patients to predict the disease or symptoms of the disease.

Another example use case applies the technology disclosed to an application program used by millions of members who regularly use the application for a function, leaving digital traces that reveal the members' interests in a data trail. For instance, someone may look for restaurants. In this example, a technology (or tech) company requires user feedback in order to improve the quality of its prediction model to serve users better. The tech company gives this input to FL aggregator and gets the tensors back, asynchronously, or synchronously. Doing so, the raw data of end users is not used, and privacy of end users is not invaded. The tech company only gets a global model of the interests of the entire population and a more precise model in different behavioral segments that enables them to target specific predicted actions. The company can also share either the global tensors or the precision tensors, should they want to. No data is transported, inferences can be drawn by applying the tensors, without access to underlying user data.

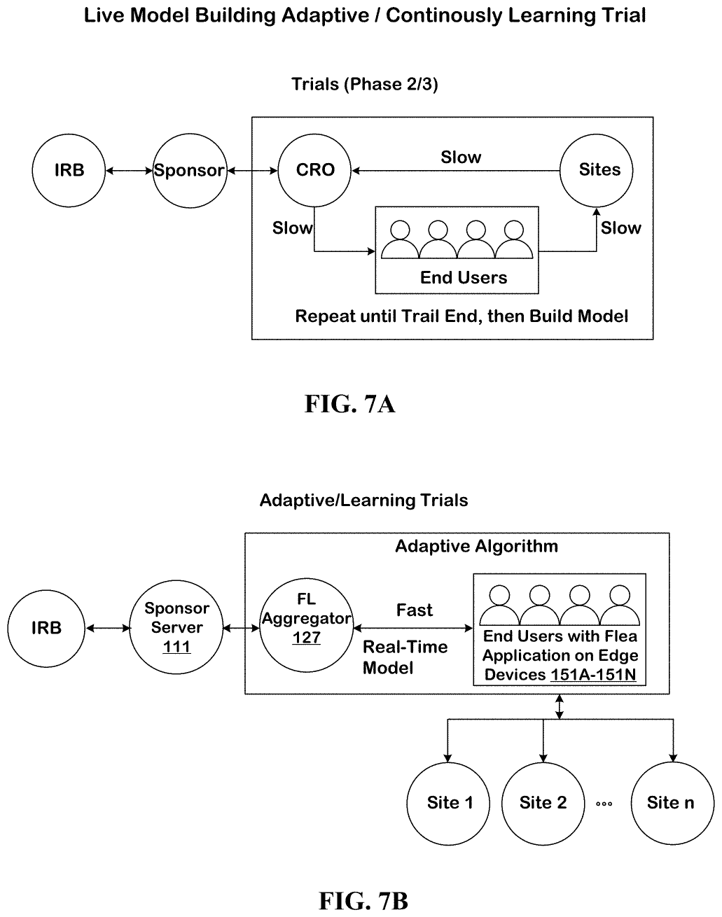

FIGS. 7A-7B are diagrams illustrating example use case of a federated learner system in a linear training trial and in an adaptive and continuously learning distributed trial, comprising federated learners and FL aggregator or the clinical trial conductor server applied to collection and analysis of data trial.

With a federated learner and clinical trial conductor server, clinical trials do not require site visits. On a site visit, CROs receive the data from the sites, which is an arduous data collection process that takes significant time. The CROs analyze the data once the trial is complete, which takes significant amount of time and money to do so. Correcting model errors is expensive, especially if a part of the trial has to be reevaluated. With federated learner, trials are in real-time, especially because end points of the trials are already being built as prediction models or analytics. Administrators can control the data training and frequency behind the scenes and it is the algorithms that are adaptive, instead of humans in a CRO. Trials are more streamlined and parallelized. Speed of trial is significantly improved, even though it may possibly mean failing fast. Feedback loops are much faster, and the sponsors or CROs get a much better idea whether the trial is even working correctly from early on.

An end user can use a site of their choice, provided the site is also chosen with the trial. The data on end user's phone is used for training the model relevant to the end point of the trial. Since the analytics and model are not an after-trial completion artifact but living and real-time with the federated learner, administrators of the trial can quickly adapt to issues of bias, confounding influences, etc. This speeds up trials. End users can be virtual or on-site. Additionally, trials can collect real world data from user devices that provides more dimensions for training. We now present an allergy symptoms prediction model that can be trained in a clinical trial using the technology disclosed.

Allergy Prediction Model

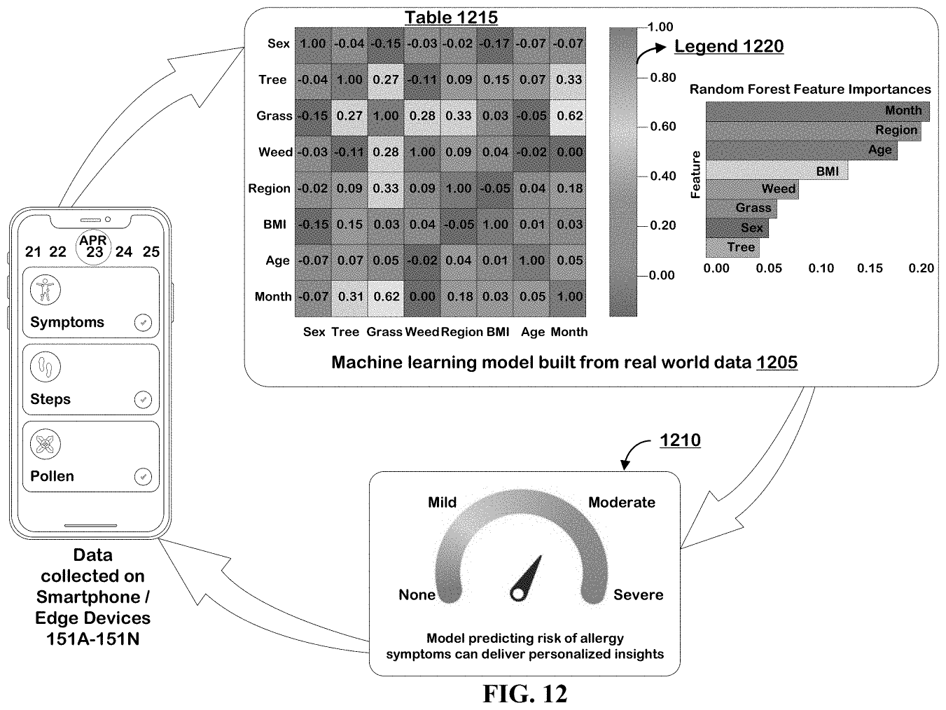

One working example of horizontal learning executed in a mobile environment is predicting risk to allergies (as shown in FIG. 12). In one implementation, the machine learning model (or clinical trial objective mapper) trained in this clinical trial is a random forest model. Other examples of clinical trial objective mappers can include neural networks such as Convolutional Neural Networks (CNNs) which are commonly used for image classification tasks. The model is trained on data collected from hundreds or more participants using the respective edge devices such as mobile or handheld devices. The technology disclosed can enroll the participants in the clinical trial via an app (for example, OMIX™ app) running on their respective edge devices.

The participants can enter the participant-specific clinical data as input in virtual clinical trial study. The input can include phenome demographics such as age, sex, height, weights, etc. The input can also include location data of the participants. The location data can be linked to pollen count at respective location or other measurements related to local environment. The participants can enter the data in the app once a day. The participants can also enter their respective symptoms such as “none”, “mild”, “moderate”, or “severe” once a day. The model can then be trained locally on each device using this participant-specific data as ground truth. Other examples of participant-specific clinical data (or input) can include an image such as a selfie image of the participant or an image of prescription medicine container, etc. Participant-specific clinical data can also include an audio recording such as that of the participant. Data from apps, fitness trackers or medical devices can also be given as input to the clinical trial objective mapper. Such data can be passively collected by the system once data collection is approved by the participant. The participant-specific clinical data can also include historical clinical data such as from previous clinical trials or studies in which the participant has participated or from medical or laboratory records such as Electronic Health Records, laboratory reports, insurance claims, prescription records, etc.

The machine learning model, or the clinical trial objective mapper can be deployed on respective edge devices of participants via an app. The app requests participants of the clinical trial to use it as a daily prediction about their risk of allergies. This model can be trained in a federated manner, beginning with a base model 1051 trained conventionally to produce a model that performs relatively well. This base model is sent to an edge device where it's first used to perform inference on new data collected by the user, such as age, sex, height, weight, and pollen count based on the location data. The user will be given the option to correct the allergy inferences made by the model, so that accurate predictions are known. The participants can provide input whether the allergy prediction from the model is correct or incorrect. The participant can also provide additional input such as allergy symptoms as a text or on a scale such as mild, moderate, or severe. The system can use the input to update the model's gradients or parameters. With this ground truth, the base model is trained to produce an updated model. Each of the participating edge devices similarly produces local updates to the current model. Those local updates are centrally aggregated into a new based model and the process repeats. The updated gradients (or tensors) are then sent to the clinical trial conductor server (or Flea Circus). Therefore, the prediction accuracy of the model can be improved without any participant-specific data leaving the respective edge devices of the participants. The clinical trial conductor server can aggregate the participant-specific gradients that can cumulatively improve the model predictions. The updated gradients can then be shared with participants of the clinical trial to repeat the training.

The clinical trial objective mapper maps inputs to target mapping which can be a prediction of allergy risk such as “none”, “mild”, “moderate”, or “severe”. A trained clinical trial objective mapper 1205 can be used to predict a disease or symptoms of a disease, efficacy of a treatment, drug or therapy, health anomaly such as when patient or end user's input data is out bounds or out of range compared with participants of the clinical trial. The model prediction can be displayed using a prediction interface 1210.

The values in the table 1215 in FIG. 12 represents correlation coefficients between respective values of features in rows and columns. The higher correlation, the closer the color is to green. The lower the correlation, the closer the color is to red. A color legend 1220 maps the correlation values to colors. The highest correlation value of 1 corresponds to green color at the top of the vertical bar. The lowest correlation value of 0 corresponds to red color at the bottom. As shown in the table 1215 for example, there is a high correlation of 0.62 between “month” and “grass” features. The illustration 1205 of the machine learning model also shows importance values of random forest features. “Month” feature has the highest value of 0.20 whereas “tree” feature has the lowest importance value.

Aggregation can be performed using a federated average algorithm. Examples of aggregation can include a weighted average of the updates to the model, weighted according to the number of samples used by an edge device to produce its update. Alternatively, only updates based on a threshold number of samples would be aggregated and the aggregation could be un-weighted.

Initial training of the base model can be offline. Then, the trained base model can be distributed to edge devices, which produce updates that are processed by the federated loop, as illustrated in FIG. 10. The engineering challenges are significant. One challenge arises from networking issues and latency of devices are joining and leaving the network. Another challenge is that the mobile model is unquantized and includes on the order 20 megabytes of model parameters. It is useful to make sure that the model is not updated too often over cellular data connections. Updating also hits the mobile device's power constraints, as training on a mobile phone is resource intensive and, therefore, power hungry. In some implementations, training is limited to times when the phone is plugged in, has a Wi-Fi connection and is not in otherwise use by the user.

On the server side, asynchronous task management requires record keeping and keeping track of all of the training tasks and local updates in process numerous edge device. It also involves periodically performing aggregation and redeploying updated models. In addition to these engineering challenges, there are theoretical concerns, arising from classical statistics, that can only be overcome by empirical investigation.

Technology disclosed includes logic to apply federated learning (FL) to subtasks (or subparts) of a clinical trial. The subparts can be related to various tasks in a clinical trial. For example, data collection tasks are carried out manually in traditional clinical trials. The system can train clinical trial objective mapper for a particular type of data so that the system can automatically predict the required data using readily available inputs on respective edge devices. Consider a clinical trial that requires collection of body mass index (BMI) and weight of participants in a clinical trial. If this task is carried out manually, the participants in the clinical trial enter their respective data manually or the edge devices receive the data from one or more sensors.

The technology disclosed can deploy a machine learning model on edge devices 151A-151N that can predict the required data (such as weight and BMI) from selfie images of respective participants. The model can continue to train in the background on edge devices and only send weights of the model (or gradients) to the clinical trial conductor server 127. Therefore, the system does not send sensitive information such as participant's selfie image to the clinical trial conductor server. Similar machine learning models can be deployed to edge devices in the example of allergy prediction model clinical trial. One or more machine learning models can be deployed to edge devices for data collection and other tasks without sharing participant's data to other devices or servers.

Further Applications Related to Clinical Trials

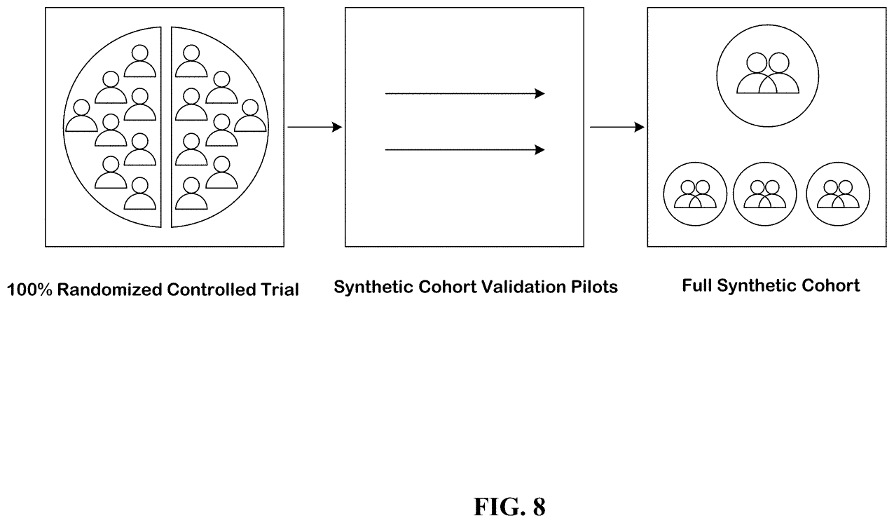

FIG. 8 is a diagram illustrating an example use case of a federated learner system, including one or more simulated control arms for the application of data trial. So-called synthetic control arms are configured to operate via collected data at large scale over an existing population. Synthetic control arms can save time and money in clinical trials. The same populations can be used to train generative models for similar populations. These generative models can cause a many-fold increase in the utility of the population based on its simulated characteristics.

Instead of collecting data from patients recruited for a trial who have been assigned to the control or standard-of-care arm, synthetic control arms model those comparators using real-world data that has previously been collected from sources such as health data generated during routine care, including electronic health records, administrative claims data, patient-generated data from fitness trackers or home medical equipment, disease registries, and historical clinical trial data, etc. This can be done via a federated learning model with edge devices sending up gradients to at least one FL aggregator.

Synthetic control arms bring clear benefits to pharmaceutical industry and application. It can reduce or even eliminate to enroll control end users, improve efficiency, efficacy and consistency. By reducing or eliminating the need to enroll control end users, a synthetic control arm can increase efficiency, reduce delays, lower trial costs, and speed up life-saving therapies to market. This kind of hybrid trial design presents a less risky way for sponsors to introduce real-world data elements into regulatory trials and can also reduce the risk of late-stage failures by informing go or no-go development decisions. Placebo-fear is one of the top-reasons patients choose not to participate in clinical trials. This concern is amplified when an individual's prognosis is poor and when current care is of limited effectiveness. Using a synthetic control arm instead of a standard control arm ensures that all participants receive the active treatment, eliminating concerns about treatment/placebo assignment. Use of a synthetic control arm addresses an important participant concerns and removes an important barrier to recruitment. The use of simulated control arms can also eliminate the risk of unblinding when patients lean on their disease support social networks posting details of their treatment, progress, and side effects that could harm the integrity of the trial.

The federated learner system can be utilized for tensorial twins or phenotype twins. The tensorial twin represents the nearest-neighbor patient, derived from algorithmic matching of the maximal proportion of data points using a subtype of AI known as nearest-neighbor analysis. The nearest neighbor is identified using AI analytics for approximating a facsimile, another human being as close as possible to an exact copy according to the patient's characteristics to help inform best treatment, outcomes, and even prevention.

We can use information that comprehensively characterizes each individual for demographics, biologic omics, physiology, anatomy, and environment, along with treatment and outcomes for medical conditions.

Perturbed Subspace Method (PSM) employs a predicted probability of group membership, e.g., treatment or control group, based on observed predictors, usually obtained from logistic regression to create a counterfactual group. Propensity scores may also be used for matching or as covariates—alone or with other matching variables or covariates. With federated learning every cohort can be configured to be adaptive in a very complex way because the members with federated learner could send up delta. In this case, it continuously makes the relationship between them and the cohort tenuous to the point that they redefine normality and start to act as patients in silico, preparing for a stochastic forward model of precision medicine.

The federated learner system may use fuzzy tensor swarm. Devices which used to be responsible only for the gathering of data are to be configured to run downstream computations. Such configuration can be applied to various scenarios. For example, heart rate monitors, automatic blood pressure pumps, weather micro-stations, etc. Computational capacity as well as speed are increased drastically. With the advent of higher-bandwidth connectivity between such devices (due, for example, to 5G), the old paradigm of requiring these devices to send data to a central location where an archaic batch runner produces an updated data processor and ships it back to each device individually is becoming outmoded. Incurring a system-wide overhead when heart rate monitor can update its own data processing algorithms makes no sense. Such heart rate monitor system only requires access blood pressure pump and weather micro-station.

As in the case of the heart rate monitor, the capability of updating the system's own data processing algorithm by the system itself is especially true for mission-critical functionality, where seconds could make a difference between life and death. To make use of this additional computational capacity and bandwidth, each device is to be deployed with its own adaptive data processing module, placed within a network mesh of devices, and equipped with an ontology (e.g., protocol-driven) describing to it the kind of information it can derive from each of its neighbors in the mesh. Each device in the mesh is configured to make available to its neighbors any of its primitives, as well as data-derived updates to itself. Taken together, an ensemble of interconnected devices, of which each with an intelligent data processing module and an ontological protocol, form a fuzzy tensor swarm. In this fuzzy tensor swarm, the emergent behavior is configured at a minimum equivalent in functionality, although may not be optimal in terms of latency and overhead, to what is possible with a centralized model building workflow. Empowered by 5G and Internet-of-Things technologies, each device can be connected, either physically or not, and stream data to millions of other smart data capture devices that can create live models of their vertical worlds. The enriched information from millions of graphics processing units can be feedbacked to other objects or their carbon, silicon or neuron users. Passive collection can be monetized and become the service industry of virtual reality (VR) which can create parallel existential dimensions as a service.

In some embodiments of the disclosure, a federated learner model can be applied to federated learning and adversarial rapid testing of clinical data and standards. Data training done on the device close to the data mitigates privacy concerns. The trained models basically try to predict when symptoms happen, and the user can be enabled to verify. This Generative Adversarial Network (GAN) can then be used to generate Real World Evidence (RWE) backed patient simulations to validate clinical trials, data, anomaly detection. Pharmaceutical company can be enabled to license these models out as new revenue. End users' simulated data is predicted or inferred on probabilistic risk calculators, based on their genetics, exposome, pharmacome and other omics data. Once these models are built, pharmaceutical company can also use the models in other data trials to do groundwork analysis.

Clinical trial can go out with consumer health care mobile devices, e.g., a fitness tracker or a wearable device, where participants can confirm or deny when the GAN thinks they may have a symptom happen soon. The model gets trained on end user devices and only the model is sent back to the servers. The models are then tested in other patients and verified over and over.

This model of symptoms can be used to simulate existing clinical trial around similar drug. If it can reproduce the study results, then these models can be used in dashboard around these types of drugs.

The federated learning model can be applied to automatic qualification of participants for clinical trials and remove the expensive human verification process.

The federated learning model can be applied to decentralized patient registries. Such registry is on the edge and fragmented but comes together on an “ask” command by authorized personnel, e.g., the end user.

The federated learning model can be applied to configure peer to peer health data comparator to compare health condition of one end user against another without sharing any personal data.

The federated learning model can be applied to distribute second opinion. One end user can be enabled to share his or her personal model with a new doctor or citizen scientist without giving away any data. Tensors are compared and not the real data.

The federated learning model can be applied to health anomaly detection via model anomaly detection. Tensors can be configured to indicate that there is an out of bounds anomaly with the population. Once some issues identified, it can escalate to a doctor.

The federated learning model can be applied to health fingerprint. The model built on end user data can be a unique signature of the end user. It evolves as the health condition of the end user evolves. The model can be used as an identity in time.

In yet some other embodiments of the disclosure, there are many-to-many tensors for distributed clinical trials. Just as algorithms start to write themselves, devices are configured to collect information between each other without human intervention. Cheap Micro-Computer Units (MCUs) can soon be deployed anywhere, without mains, docking, or battery replacement. MCUs can be configured to behave like many insect species, including ants and bees, who work together in colonies. The cooperative behavior of the group of MCUs determines the survival of the entire group. The group operates like a single organism, with each individual in a colony acting like a cell in the body and becomes a “superorganism.” Federated deep learning algorithm requires these small players like insects, ants, critters and bees to create big and smart things with immense, complex and adaptive social power and ambitious missions.

### Overall Approach

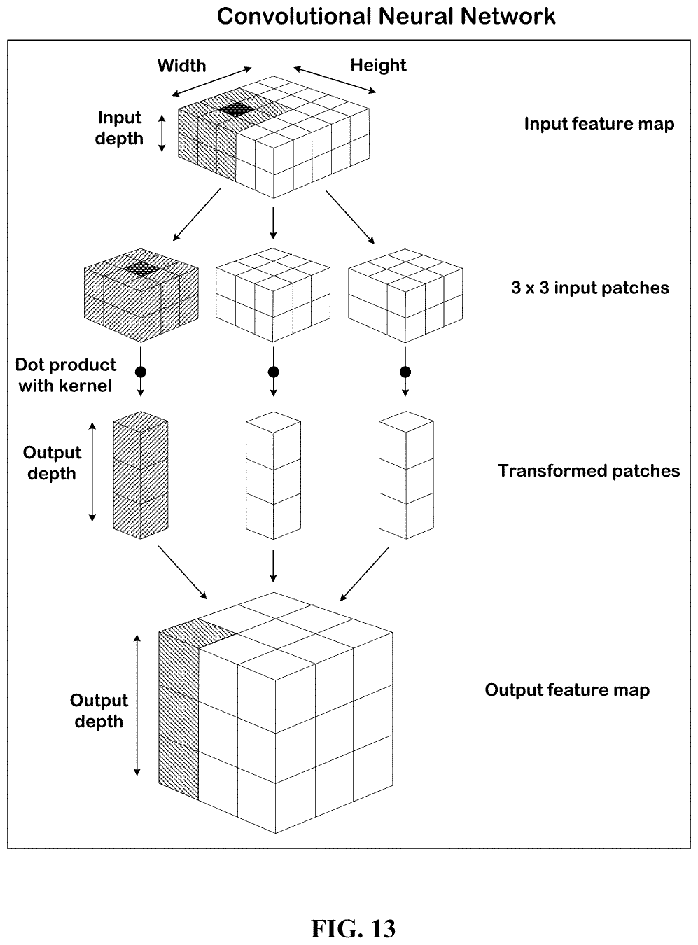

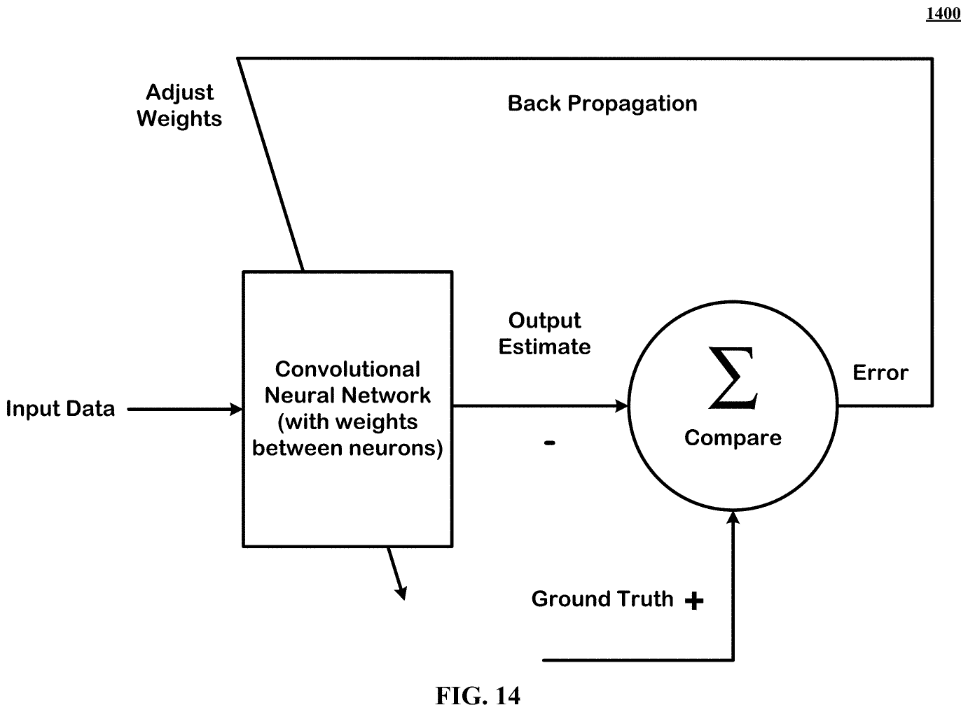

With this example in mind, we return to describing the overall approach. As shown in FIG. 1, Flea end users or participants, via respective edge devices, can communicate and collaborate with one another (potentially in tandem with one or more FL aggregator backends) to build and update models of computation in multiple ways. These configurations are described in the context of medical research use cases. We present a general discussion of random forest machine learning technique as a first example of machine learning models that can be used by the technology disclosed. A general discussion regarding convolutional neural networks, CNNs, and training by gradient descent is presented as a second example of a machine learning model that can be used by the technology disclosed. The discussion of CNNs is facilitated by FIGS. 13-14.

Random Forest Model

Random forest classifier (also referred to as random decision forest) is an ensemble machine learning technique. Ensemble techniques or algorithms combine more than one technique of the same or different kind for classifying objects. The random forest classifier consists of multiple decision trees that operate as an ensemble. Each individual decision tree in random forest acts as a base classifier and outputs a class prediction. The class with the most votes becomes the random forest model's prediction. The fundamental concept behind random forests is that a large number of relatively uncorrelated models (decision trees) operating as a committee will outperform any of the individual constituent models.

Random Forest is an ensemble machine learning technique based on bagging. In bagging-based techniques, during training, subsamples of records are used to train different models such as decision trees in random forest. In addition, feature subsampling can also be used. The idea is that different models will be trained on different types of features and therefore, overall, the model will perform well in production. The output of random forest is based on the output of individual models such as decision trees. The output from individual models is combined to produce the output from the random forest model.

Decision trees are prone to overfitting. To overcome this issue, bagging technique is used to train the decision trees in random forest. Bagging is a combination of bootstrap and aggregation techniques. In bootstrap, during training, we take a sample of rows from our training database and use it to train each decision tree in the random forest. For example, a subset of features for the selected rows can be used in training of decision tree 1. Therefore, the training data for decision tree 1 can be referred to as row sample 1 with column sample 1 or RS1+CS1. The columns or features can be selected randomly. The decision tree 2 and subsequent decision trees in the random forest are trained in a similar manner by using a subset of the training data. Note that the training data for decision trees is generated with replacement i.e., same row data can be used in training of multiple decision trees.

The second part of bagging technique is the aggregation part which is applied during production. Each decision tree outputs a classification for each class. In case of binary classification, it can be 1 or 0. The output of the random forest is the aggregation of outputs of decision trees in the random forest with a majority vote selected as the output of the random forest. By using votes from multiple decision trees, a random forest reduces high variance in results of decision trees, thus resulting in good prediction results. By using row and column sampling to train individual decision trees, each decision tree becomes an expert with respect to training records with selected features.

During training, the output of the random forest is compared with ground truth labels and a prediction error is calculated. During backward propagation, the weights or coefficients of the model are adjusted so that the prediction error is reduced. We present details of training and hyperparameters for an example machine learning model that can used by the technology disclosed.

Model Algorithm: Random Forest with Class Weighting
Training: The cleaned dataset consisted of 10,603 rows and 9 columns. Each row represented an allergy event with level of severity as the predictor (dependent variable) and 8 independent variables (month, age class, BMI class, sex, AAAAI region, tree BPI, weed BPI, grass BPI). Input samples were shuffled and split into training and test sets at a ratio of 80/20 with stratification. Test samples were left out of all model training steps and only used for final parameter validation.

### Hyperparameters:

Number of trees: 65
Tree depth: 11
Minimum samples at each split: 2
Maximum features: 4

CNNs

A convolutional neural network is a special type of neural network. The fundamental difference between a densely connected layer and a convolution layer is this: Dense layers learn global patterns in their input feature space, whereas convolution layers learn local patterns: in the case of images, patterns found in small 2D windows of the inputs. This key characteristic gives convolutional neural networks two interesting properties: (1) the patterns they learn are translation invariant and (2) they can learn spatial hierarchies of patterns.

Regarding the first, after learning a certain pattern in the lower-right corner of a picture, a convolution layer can recognize it anywhere: for example, in the upper-left corner. A densely connected network would have to learn the pattern anew if it appeared at a new location. This makes convolutional neural networks data efficient because they need fewer training samples to learn representations, they have generalization power.

Regarding the second, a first convolution layer can learn small local patterns such as edges, a second convolution layer will learn larger patterns made of the features of the first layers, and so on. This allows convolutional neural networks to efficiently learn increasingly complex and abstract visual concepts.

A convolutional neural network learns highly non-linear mappings by interconnecting layers of artificial neurons arranged in many different layers with activation functions that make the layers dependent. It includes one or more convolutional layers, interspersed with one or more sub-sampling layers and non-linear layers, which are typically followed by one or more fully connected layers. Each element of the convolutional neural network receives inputs from a set of features in the previous layer. The convolutional neural network learns concurrently because the neurons in the same feature map have identical weights. These local shared weights reduce the complexity of the network such that when multi-dimensional input data enters the network, the convolutional neural network avoids the complexity of data reconstruction in feature extraction and regression or classification process.

Convolutions operate over 3D tensors, called feature maps, with two spatial axes (height and width) as well as a depth axis (also called the channels axis). For an RGB image, the dimension of the depth axis is 3, because the image has three color channels; red, green, and blue. For a black-and-white picture, the depth is 1 (levels of gray). The convolution operation extracts patches from its input feature map and applies the same transformation to all of these patches, producing an output feature map. This output feature map is still a 3D tensor: it has a width and a height. Its depth can be arbitrary, because the output depth is a parameter of the layer, and the different channels in that depth axis no longer stand for specific colors as in RGB input; rather, they stand for filters. Filters encode specific aspects of the input data: at a height level, a single filter could encode the concept “presence of a face in the input,” for instance.

For example, the first convolution layer takes a feature map of size (28, 28, 1) and outputs a feature map of size (26, 26, 32): it computes 32 filters over its input. Each of these 32 output channels contains a 26×26 grid of values, which is a response map of the filter over the input, indicating the response of that filter pattern at different locations in the input. That is what the term feature map means: every dimension in the depth axis is a feature (or filter), and the 2D tensor output [:; :, n] is the 2D spatial map of the response of this filter over the input.

Convolutions are defined by two key parameters: (1) size of the patches extracted from the inputs—these are typically 1×1, 3×3 or 5×5 and (2) depth of the output feature map—the number of filters computed by the convolution. Often these start with a depth of 32, continue to a depth of 64, and terminate with a depth of 128 or 256.

A convolution works by sliding these windows of size 3×3 or 5×5 over the 3D input feature map, stopping at every location, and extracting the 3D patch of surrounding features (shape (window_height, window_width, input_depth)). Each such 3D patch is ten transformed (via a tensor product with the same learned weight matrix, called the convolution kernel) into a 1D vector of shape (output_depth,). All of these vectors are then spatially reassembled into a 3D output map of shape (height, width, output_depth). Every spatial location in the output feature map corresponds to the same location in the input feature map (for example, the lower-right corner of the output contains information about the lower-right corner of the input). For instance, with 3×3 windows, the vector output [i, j, :] comes from the 3D patch input [i−1: i+1, j−1:J+1, :].

The convolutional neural network comprises convolution layers which perform the convolution operation between the input values and convolution filters (matrix of weights) that are learned over many gradient update iterations during the training. Let (m, n) be the filter size and W be the matrix of weights, then a convolution layer performs a convolution of the W with the input X by calculating the dot product W·x+b, where x is an instance of X and b is the bias. The step size by which the convolution filters slide across the input is called the stride, and the filter area (m×n) is called the receptive field. A same convolution filter is applied across different positions of the input, which reduces the number of weights learned. It also allows location invariant learning, i.e., if an important pattern exists in the input, the convolution filters learn it no matter where it is in the sequence.

Training a Convolutional Neural Network

FIG. 14 depicts a block diagram 1400 of training a convolutional neural network in accordance with one implementation of the technology disclosed. The convolutional neural network is adjusted or trained so that the input data leads to a specific output estimate. The convolutional neural network is adjusted using back propagation based on a comparison of the output estimate and the ground truth until the output estimate progressively matches or approaches the ground truth.

The convolutional neural network is trained by adjusting the weights between the neurons based on the difference between the ground truth and the actual output. This is mathematically described as:

$$Δ {w}_{i} = {x}_{i} δ$$

$$where δ = ( ground truth ) - ( actual output )$$

In one implementation, the training rule is defined as:

Wₙₘ←Wₙₘ+α(tₘ−φₘ)aₙ

In the equation above: the arrow indicates an update of the value; tₘ is the target value of neuron m; φₘ is the computed current output of neuron m; aₙ is input n; and α is the learning rate.

The intermediary step in the training includes generating a feature vector from the input data using the convolution layers. The gradient with respect to the weights in each layer, starting at the output, is calculated. This is referred to as the backward pass, or going backwards. The weights in the network are updated using a combination of the negative gradient and previous weights.

In one implementation, the convolutional neural network uses a stochastic gradient update algorithm (such as ADAM) that performs backward propagation of errors by means of gradient descent. One example of a sigmoid function based back propagation algorithm is described below:

$$ϕ = f ( h ) = \frac{1}{1 + {e}^{- h}}$$

In the sigmoid function above, h is the weighted sum computed by a neuron. The sigmoid function has the following derivative:

$$\frac{\partial ϕ}{\partial h} = ϕ ( 1 - ϕ )$$

The algorithm includes computing the activation of all neurons in the network, yielding an output for the forward pass. The activation of neuron m in the hidden layers is described as:

$${ϕ}_{m} = \frac{1}{1 + {e}^{- hm}}$$

$${h}_{m} = {\sum }_{n = 1}^{N} {a}_{n} {w}_{nm}$$

This is done for all the hidden layers to get the activation described as:

$${ϕ}_{k} = \frac{1}{1 + {e}^{- hk}}$$

$${h}_{k} = {\sum }_{m = 1}^{M} {ϕ}_{m} {w}_{mk}$$

Then, the error and the correct weights are calculated per layer. The error at the output is computed as:

δₒₖ=(tₖ−φₖ)φₖ(1−φₖ)

The error in the hidden layers is calculated as:

$${δ}_{h m} = {ϕ}_{m} ( 1 - {ϕ}_{m} ) {\sum }_{k = 1}^{K} {v}_{m k} {δ}_{o k}$$

The weights of the output layer are updated as:

vmk←Vmk+αδokφm

The weights of the hidden layers are updated using the learning rate a as:

vnm←wnm+αδhmαn

In one implementation, the convolutional neural network uses a gradient descent optimization to compute the error across all the layers. In such an optimization, for an input feature vector x and the predicted output ŷ, the loss function is defined as l for the cost of predicting ŷ when the target is y, i.e., l(ŷ, y). The predicted output ŷ is transformed from the input feature vector x using function ƒ. Function ƒ is parameterized by the weights of convolutional neural network, i.e., ŷ=ƒ_{w}(x). The loss function is described as l(ŷ, y)=l(ƒ_{w}(x), y), or

Q(z, w)=l(ƒ_{w}(x), y) where z is an input and output data pair (x, y). The gradient descent optimization is performed by updating the weights according to:

$${v}_{t} + 1 = μ {v}_{t} - α \frac{1}{n} {\sum }_{i = 1}^{N} ∇ wt Q ( zt , Wt )$$

$${w}_{t + 1} = {w}_{t} + {v}_{t + 1}$$

In the equations above, α is the learning rate. Also, the loss is computed as the average over a set of n data pairs. The computation is terminated when the learning rate α is small enough upon linear convergence. In other implementations, the gradient is calculated using only selected data pairs fed to a Nesterov's accelerated gradient and an adaptive gradient to inject computation efficiency.

In one implementation, the convolutional neural network uses a stochastic gradient descent (SGD) to calculate the cost function. An SGD approximates the gradient with respect to the weights in the loss function by computing it from only one, randomized, data pair, Zₜ, described as:

vₜ+μv−α∇wQ(zₜ,wₜ)

wₜ₊₁=wₜ+vₜ₊₁

In the equations above: α is the learning rate; μ is the momentum; and tis the current weight state before updating. The convergence speed of SGD is approximately O(1/t) when the learning rate α are reduced both fast and slow enough. In other implementations, the convolutional neural network uses different loss functions such as Euclidean loss and softmax loss. In a further implementation, an Adam stochastic optimizer is used by the convolutional neural network.

### Particular Implementations

We describe implementations of a system to conduct virtual clinical trials.

The technology disclosed can be practiced as a system, method, or article of manufacture. One or more features of an implementation can be combined with the base implementation. Implementations that are not mutually exclusive are taught to be combinable. One or more features of an implementation can be combined with other implementations. This disclosure periodically reminds the user of these options. Omission from some implementations of recitations that repeat these options should not be taken as limiting the combinations taught in the preceding sections—these recitations are hereby incorporated forward by reference into each of the following implementations.

A system implementation of the technology disclosed includes one or more processors coupled to memory. The memory can be loaded with instructions to conduct virtual clinical trials. The system can comprise a sponsor server coupled to a communication network. The sponsor server can be configured to specify a target mapping of a clinical trial objective mapper. The target mapping maps participant-specific clinical data to an objective of a virtual clinical trial. The system comprises a plurality of edge devices coupled to the communication network. The plurality of edge devices are accessible by respective participants in a plurality of participants. The system comprises a clinical trial conductor server coupled to the communication network. The clinical trial conductor server can be interposed between the sponsor server and the plurality of edge devices. The clinical trial conductor server can distribute coefficients of the clinical trial objective mapper to respective edge devices in the plurality of edge devices to implement distributed training of the clinical trial objective mapper. The clinical trial conductor server can receive participant-specific gradients from the respective edge devices. The participant-specific gradients can be generated during the distributed training in response to processing participant-specific clinical data through the coefficients of the clinical trial objective mapper at the respective edge devices. The clinical trial conductor server can aggregate the participant-specific gradients to generate aggregated gradients that cumulatively satisfy the target mapping of the clinical trial objective mapper.

This system implementation and other systems disclosed optionally include one or more of the following features. System can also include features described in connection with methods disclosed. In the interest of conciseness, alternative combinations of system features are not individually enumerated. Features applicable to systems, methods, and articles of manufacture are not repeated for each statutory class set of base features. The reader will understand how features identified in this section can readily be combined with base features in other statutory classes.

In one implementation, the clinical trial conductor is further configured to apply aggregated gradients to coefficients of the clinical trial objective mapper to generate updated coefficients of the clinical trial objective mapper.

The participant-specific clinical data can be an image captured by the respective edge device of the participant. The participant-specific clinical data can be an audio recording of the participant captured by the respective edge device of the participant. The participant-specific clinical data can include data generated by a fitness tracker. The participant-specific clinical data includes data can be generated by a home medical equipment. The participant-specific clinical data can include participant characteristics including age, height, and weight. The participant-specific clinical data can include historical clinical trials data. The participant-specific clinical data can include location data, environmental data, climate data collected from edge devices of participants.

The clinical trial objective mapper can be a convolutional neural network. The clinical trial objective mapper can be a random forest model. The technology disclosed can use other types of machine learning techniques such as gradient boosted trees, extreme gradient boosting, etc.

In one implementation, the sponsor server can be configured to apply the clinical trial objective mapper with updated coefficients to map participant-specific clinical data to a clinical trial objective prediction. The clinical trial objective prediction can be a score indicating efficacy of a treatment. The clinical trial objective prediction can be a score indicating likelihood of a disease. The clinical trial objective prediction can include predicting symptoms of a disease. The clinical trial objective prediction can include a health anomaly detection indicating that patient's health indicators are out of bound compared with participants of the clinical trial.

In one implementation, the sponsor server is further configured to specify a target mapping of a second clinical trial objective mapper. The target mapping of the second clinical trial objective mapper can map participant-specific clinical trial data to a subtask prediction of the virtual clinical trial. The clinical trial conductor server is further configured to distribute coefficients of the second clinical trial objective mapper to respective edge devices in the plurality of edge devices to implement distributed training of the second clinical trial objective mapper. The second clinical trial objective mapper can perform the subtask prediction at respective edge devices. The clinical trial conductor server is further configured to receive participant-specific gradients from the respective edge devices. The participant-specific gradients are generated during the distributed training in response to processing participant-specific clinical data through the coefficients of the second clinical trial objective mapper at the respective edge devices. The clinical trial conductor server is further configured to aggregate the participant-specific gradients to generate aggregated gradients that cumulatively satisfy the target mapping of the second clinical trial objective mapper.

The participant-specific clinical data can be a selfie image of the participant captured by the respective edge device of the participant. The subtask prediction of the virtual clinical trial can be body weight (or weight) of the participant. The subtask prediction of the virtual clinical trial is a body mass index (BMI) of the participant.

Other implementations consistent with this system may include a non-transitory computer readable storage medium storing instructions executable by a processor to perform functions of the system described above. Yet another implementation may include a method performing the functions of the system described above.

Aspects of the technology disclosed can be practiced as a method of conducting virtual clinical trials. The method can include receiving a target mapping of a clinical trial objective mapper. The target mapping maps participant-specific clinical data to an objective of a virtual clinical trial. The method can include distributing coefficients of the clinical trial objective mapper to respective edge devices in a plurality of edge devices to implement distributed training of the clinical trial objective mapper. The edge devices in the plurality of edge devices can be accessible by respective participants in a plurality of participants. The method can include receiving participant-specific gradients from the respective edge devices. The participant-specific gradients can be generated during the distributed training in response to processing participant-specific clinical data through the coefficients of the clinical trial objective mapper at the respective edge devices. The method can include aggregating the participant-specific gradients to generate aggregated gradients that cumulatively satisfy the target mapping of the clinical trial objective mapper.

This method implementation can incorporate any of the features of the system described immediately above or throughout this application that apply to the method implemented by the system. In the interest of conciseness, alternative combinations of method features are not individually enumerated. Features applicable to systems, methods, and articles of manufacture are not repeated for each statutory class set of base features. The reader will understand how features identified in this section for one statutory class can readily be combined with base features in other statutory classes.

Other implementations consistent with this method may include a non-transitory computer readable storage medium storing instructions executable by a processor to perform any of the methods described above. Yet another implementation may include a system with memory loaded from a computer readable storage medium with program instructions to perform the method described above. The system can be loaded from either a transitory or a non-transitory computer readable storage medium.

As an article of manufacture, rather than a method, a non-transitory computer readable medium (CRM) can be loaded with program instructions executable by a processor. The program instructions when executed, implement the computer-implemented method described above. Alternatively, the program instructions can be loaded on a non-transitory CRM and, when combined with appropriate hardware, become a component of one or more of the computer-implemented systems that practice the method disclosed.

Each of the features discussed in this particular implementation section for the method implementation apply equally to CRM implementation. As indicated above, all the method features are not repeated here, in the interest of conciseness, and should be considered repeated by reference.

### Clauses

1. A federated learning system to conduct virtual clinical trials, comprising:
a sponsor server, coupled to a communication network, configured to specify a target mapping of a clinical trial objective mapper, wherein the target mapping maps participant-specific clinical data to an objective of a virtual clinical trial;
a plurality of edge devices, coupled to the communication network, and accessible by respective participants in a plurality of participants; and
a clinical trial conductor server, coupled to the communication network, interposed between the sponsor server and the plurality of edge devices, and configured to:
   distribute coefficients of the clinical trial objective mapper to respective edge devices in the plurality of edge devices to implement distributed training of the clinical trial objective mapper,
   receive, from the respective edge devices, participant-specific gradients generated during the distributed training in response to processing participant-specific clinical data through the coefficients of the clinical trial objective mapper at the respective edge devices,
   aggregate the participant-specific gradients to generate aggregated gradients that cumulatively satisfy the target mapping of the clinical trial objective mapper.
      2. The system of clause 1, further comprising, the clinical trial conductor server further configured to apply aggregated gradients to coefficients of the clinical trial objective mapper to generate updated coefficients of the clinical trial objective mapper.
      3. The system of clause 1, wherein the participant-specific clinical data is an image captured by the respective edge device of the participant.
      4. The system of clause 1, wherein the participant-specific clinical data is an audio recording of the participant captured by the respective edge device of the participant.
      5. The system of clause 1, wherein the participant-specific clinical data includes data generated by a fitness tracker.
      6. The system of clause 1, wherein the participant-specific clinical data includes data generated by a home medical equipment.
      7. The system of clause 1, wherein the participant-specific clinical data includes participant characteristics including age, height, and weight.
      8. The system of clause 1, wherein the participant-specific clinical data includes historical clinical trials data.
      9. The system of clause 1, wherein the clinical trial objective mapper is a convolutional neural network.
      10. The system of clause 1, further comprising, the sponsor server configured to apply the clinical trial objective mapper with updated coefficients to map participant-specific clinical data to a clinical trial objective prediction.
      11. The system of clause 10, wherein the clinical trial objective prediction is a score indicating efficacy of a treatment.
      12. The system of clause 10, wherein the clinical trial objective prediction is a score indicating likelihood of a disease.
      13. The system of clause 10, wherein the clinical trial objective prediction predicts symptoms of a disease.
      14. The system of clause 10, wherein the clinical trial objective prediction is health anomaly detection indicating patient's health indicators are out of bound compared with participants of the clinical trial.
      15. The system of clause 1, further comprising:
the sponsor server further configured to specify a target mapping of a second clinical trial objective mapper wherein the target mapping of the second clinical trial objective mapper maps participant-specific clinical trial data to a subtask prediction of the virtual clinical trial;
the clinical trial conductor server further configured to:
   distribute coefficients of the second clinical trial objective mapper to respective edge devices in the plurality of edge devices to implement distributed training of the second clinical trial objective mapper to perform the subtask prediction at respective edge devices,
   receive, from the respective edge devices, participant-specific gradients generated during the distributed training in response to processing participant-specific clinical data through the coefficients of the second clinical trial objective mapper at the respective edge devices, and
   aggregate the participant-specific gradients to generate aggregated gradients that cumulatively satisfy the target mapping of the second clinical trial objective mapper.
      16. The system of clause 15, wherein the participant-specific clinical data is a selfie image of the participant captured by the respective edge device of the participant.
      17. The system of clause 15, wherein the participant-specific clinical data is an image captured by the respective edge device of the participant.
      18. The system of clause 15, wherein the participant-specific clinical data is an audio recording of the participant captured by the respective edge device of the participant.
      19. The system of clause 15, wherein the participant-specific clinical data includes data generated by a fitness tracker.
      20. The system of clause 15, wherein the participant-specific clinical data includes data generated by a home medical equipment.
      21. The system of clause 15, wherein the participant-specific clinical data includes participant characteristics including age, height, and weight.
      22. The system of clause 15, wherein the participant-specific clinical data includes historical clinical trials data.
      23. The system of clause 15, wherein the subtask prediction of the virtual clinical trial is a weight of the participant.
      24. The system of clause 15, wherein the subtask prediction of the virtual clinical trial is a body mass index of the participant.
      25. A method of conducting virtual clinical trials, the method including:
receiving a target mapping of a clinical trial objective mapper, wherein the target mapping maps participant-specific clinical data to an objective of a virtual clinical trial;
distributing coefficients of the clinical trial objective mapper to respective edge devices in a plurality of edge devices to implement distributed training of the clinical trial objective mapper, wherein the edge devices in the plurality of edge devices are accessible by respective participants in a plurality of participants;
receiving, from the respective edge devices, participant-specific gradients generated during the distributed training in response to processing participant-specific clinical data through the coefficients of the clinical trial objective mapper at the respective edge devices; and
aggregating the participant-specific gradients to generate aggregated gradients that cumulatively satisfy the target mapping of the clinical trial objective mapper.
   26. A non-transitory computer readable storage medium impressed with computer program instructions to conduct virtual clinical trials, the instructions, when executed on a processor, implement a method comprising:
receiving a target mapping of a clinical trial objective mapper, wherein the target mapping maps participant-specific clinical data to an objective of a virtual clinical trial;
distributing coefficients of the clinical trial objective mapper to respective edge devices in a plurality of edge devices to implement distributed training of the clinical trial objective mapper, wherein the edge devices in the plurality of edge devices are accessible by respective participants in a plurality of participants;
receiving, from the respective edge devices, participant-specific gradients generated during the distributed training in response to processing participant-specific clinical data through the coefficients of the clinical trial objective mapper at the respective edge devices; and
aggregating the participant-specific gradients to generate aggregated gradients that cumulatively satisfy the target mapping of the clinical trial objective mapper.

### Computer System

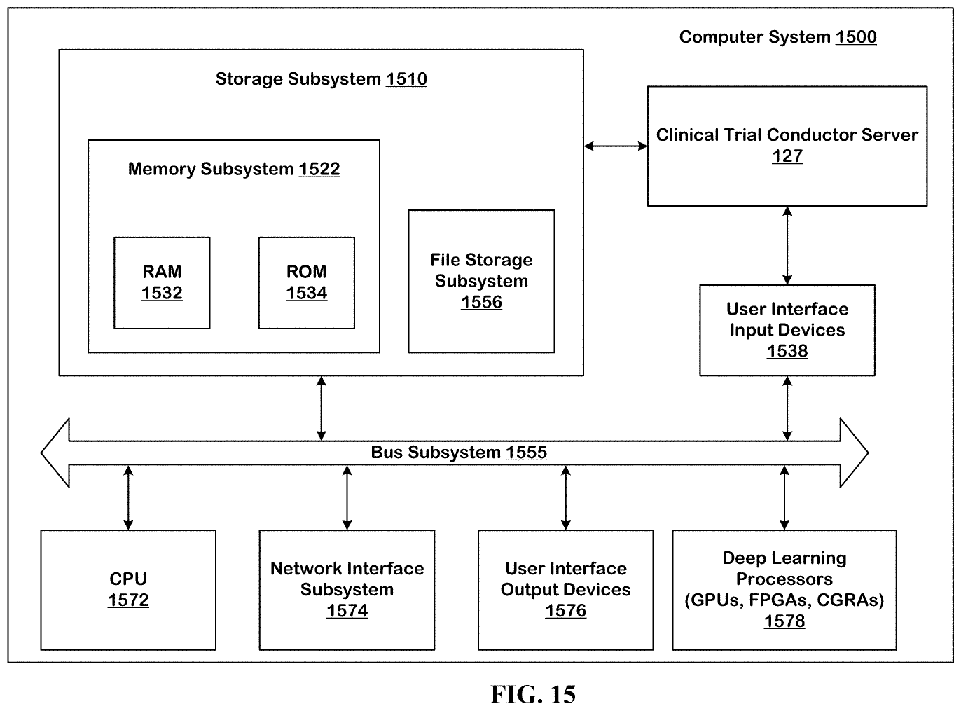

A computer-implemented method implementation of the technology disclosed includes Computer System 1500 as shown in FIG. 15.

FIG. 15 is a simplified block diagram of a computer system 1500 that can be used to implement the technology disclosed. Computer system 1500 includes at least one central processing unit (CPU) 1572 that communicates with a number of peripheral devices via bus subsystem 1555. These peripheral devices can include a storage subsystem 1510 including, for example, memory devices and a file storage subsystem 1556, user interface input devices 1538, user interface output devices 1576, and a network interface subsystem 1574. The input and output devices allow user interaction with computer system 1500. Network interface subsystem 1574 provides an interface to outside networks, including an interface to corresponding interface devices in other computer systems.

In one implementation, the clinical trial conductor server is communicably linked to the storage subsystem 1510 and the user interface input devices 1538.

User interface input devices 1538 can include a keyboard; pointing devices such as a mouse, trackball, touchpad, or graphics tablet; a scanner; a touch screen incorporated into the display; audio input devices such as voice recognition systems and microphones; and other types of input devices. In general, use of the term “input device” is intended to include all possible types of devices and ways to input information into computer system 1500.

User interface output devices 1576 can include a display subsystem, a printer, a fax machine, or non-visual displays such as audio output devices. The display subsystem can include an LED display, a cathode ray tube (CRT), a flat-panel device such as a liquid crystal display (LCD), a projection device, or some other mechanism for creating a visible image. The display subsystem can also provide a non-visual display such as audio output devices. In general, use of the term “output device” is intended to include all possible types of devices and ways to output information from computer system 1500 to the user or to another machine or computer system.

Storage subsystem 1510 stores programming and data constructs that provide the functionality of some or all of the modules and methods described herein. Subsystem 1578 can be graphics processing units (GPUs) or field-programmable gate arrays (FPGAs).

Memory subsystem 1522 used in the storage subsystem 1510 can include a number of memories including a main random access memory (RAM) 1532 for storage of instructions and data during program execution and a read only memory (ROM) 1534 in which fixed instructions are stored. A file storage subsystem 1536 can provide persistent storage for program and data files, and can include a hard disk drive, a floppy disk drive along with associated removable media, a CD-ROM drive, an optical drive, or removable media cartridges. The modules implementing the functionality of certain implementations can be stored by file storage subsystem 1556 in the storage subsystem 1510, or in other machines accessible by the processor.

Bus subsystem 1555 provides a mechanism for letting the various components and subsystems of computer system 1500 to communicate with each other as intended. Although bus subsystem 1555 is shown schematically as a single bus, alternative implementations of the bus subsystem can use multiple busses.

Computer system 1500 itself can be of varying types including a personal computer, a portable computer, a workstation, a computer terminal, a network computer, a television, a mainframe, a server farm, a widely-distributed set of loosely networked computers, or any other data processing system or user device. Due to the ever-changing nature of computers and networks, the description of computer system 1500 depicted in FIG. 15 is intended only as a specific example for purposes of illustrating the preferred embodiments of the present invention. Many other configurations of computer system 1500 are possible having more or less components than the computer system depicted in FIG. 15.

The computer system 1500 includes GPUs or FPGAs 1578. It can also include machine learning processors hosted by machine learning cloud platforms such as Google Cloud Platform, Xilinx, and Cirrascale. Examples of deep learning processors include Google's Tensor Processing Unit (TPU), rackmount solutions like GX4 Rackmount Series, GX8 Rackmount Series, NVIDIA DGX-1, Microsoft' Stratix V FPGA, Graphcore's Intelligence Processing Unit (IPU), Qualcomm's Zeroth platform with Snapdragon processors, NVIDIA's Volta, NVIDIA's DRIVE PX, NVIDIA's JETSON TX1/TX2 MODULE, Intel's Nirvana, Movidius VPU, Fujitsu DPI, ARM's DynamicIQ, IBM TrueNorth, and others.

We claim as follows:

1. A federated learning system to conduct virtual clinical trials, comprising:
a sponsor server, coupled to a communication network, configured to specify a target mapping of a clinical trial objective mapper, wherein the target mapping maps participant-specific clinical data to an objective of a virtual clinical trial;
a plurality of edge devices, coupled to the communication network, and accessible by respective participants in a plurality of participants; and
a clinical trial conductor server, coupled to the communication network, interposed between the sponsor server and the plurality of edge devices, and configured to:
distribute coefficients of the clinical trial objective mapper to respective edge devices in the plurality of edge devices to implement distributed training of the clinical trial objective mapper,
receive, from the respective edge devices, participant-specific gradients generated during the distributed training in response to processing participant-specific clinical data through the coefficients of the clinical trial objective mapper at the respective edge devices,
aggregate the participant-specific gradients to generate aggregated gradients that cumulatively satisfy the target mapping of the clinical trial objective mapper.

2. The system of claim 1, further comprising, the clinical trial conductor server further configured to apply aggregated gradients to coefficients of the clinical trial objective mapper to generate updated coefficients of the clinical trial objective mapper.

3. The system of claim 1, wherein the participant-specific clinical data is an image captured by the respective edge device of the participant.

4. The system of claim 1, wherein the participant-specific clinical data is an audio recording of the participant captured by the respective edge device of the participant.

5. The system of claim 1, wherein the participant-specific clinical data includes data generated by a fitness tracker.

6. The system of claim 1, wherein the participant-specific clinical data includes data generated by a home medical equipment.

7. The system of claim 1, wherein the participant-specific clinical data includes participant characteristics including age, height, and weight.

8. The system of claim 1, wherein the participant-specific clinical data includes historical clinical trials data.

9. The system of claim 1, wherein the clinical trial objective mapper is a convolutional neural network.

10. The system of claim 1, further comprising, the sponsor server configured to apply the clinical trial objective mapper with updated coefficients to map participant-specific clinical data to a clinical trial objective prediction.

11. The system of claim 10, wherein the clinical trial objective prediction is a score indicating efficacy of a treatment.

12. The system of claim 10, wherein the clinical trial objective prediction is a score indicating likelihood of a disease.

13. The system of claim 10, wherein the clinical trial objective prediction predicts symptoms of a disease.

14. The system of claim 10, wherein the clinical trial objective prediction is health anomaly detection indicating patient's health indicators are out of bound compared with participants of the clinical trial.

15. The system of claim 1, further comprising:
the sponsor server further configured to specify a target mapping of a second clinical trial objective mapper wherein the target mapping of the second clinical trial objective mapper maps participant-specific clinical trial data to a subtask prediction of the virtual clinical trial;
the clinical trial conductor server further configured to:
distribute coefficients of the second clinical trial objective mapper to respective edge devices in the plurality of edge devices to implement distributed training of the second clinical trial objective mapper to perform the subtask prediction at respective edge devices,
receive, from the respective edge devices, participant-specific gradients generated during the distributed training in response to processing participant-specific clinical data through the coefficients of the second clinical trial objective mapper at the respective edge devices, and
aggregate the participant-specific gradients to generate aggregated gradients that cumulatively satisfy the target mapping of the second clinical trial objective mapper.

16. The system of claim 15, wherein the participant-specific clinical data is a selfie image of the participant captured by the respective edge device of the participant.

17. The system of claim 15, wherein the subtask prediction of the virtual clinical trial is a weight of the participant.

18. The system of claim 15, wherein the subtask prediction of the virtual clinical trial is a body mass index of the participant.

19. A method of conducting virtual clinical trials, the method including:
receiving a target mapping of a clinical trial objective mapper, wherein the target mapping maps participant-specific clinical data to an objective of a virtual clinical trial;
distributing coefficients of the clinical trial objective mapper to respective edge devices in a plurality of edge devices to implement distributed training of the clinical trial objective mapper, wherein the edge devices in the plurality of edge devices are accessible by respective participants in a plurality of participants;
receiving, from the respective edge devices, participant-specific gradients generated during the distributed training in response to processing participant-specific clinical data through the coefficients of the clinical trial objective mapper at the respective edge devices; and
aggregating the participant-specific gradients to generate aggregated gradients that cumulatively satisfy the target mapping of the clinical trial objective mapper.

20. A non-transitory computer readable storage medium impressed with computer program instructions to conduct virtual clinical trials, the instructions, when executed on a processor, implement a method comprising:
receiving a target mapping of a clinical trial objective mapper, wherein the target mapping maps participant-specific clinical data to an objective of a virtual clinical trial;
distributing coefficients of the clinical trial objective mapper to respective edge devices in a plurality of edge devices to implement distributed training of the clinical trial objective mapper, wherein the edge devices in the plurality of edge devices are accessible by respective participants in a plurality of participants;
receiving, from the respective edge devices, participant-specific gradients generated during the distributed training in response to processing participant-specific clinical data through the coefficients of the clinical trial objective mapper at the respective edge devices; and
aggregating the participant-specific gradients to generate aggregated gradients that cumulatively satisfy the target mapping of the clinical trial objective mapper.
